(19) United States
(12) Patent Application Publication
(10) Pub. No.: US 2025/0093333 A1
(43) Pub. Date: Mar. 20, 2025

(54) IMMUNITY DETECTING KIT AND USING METHOD THEREOF

(71) Applicant: China Medical University, Taichung City (TW)

(72) Inventors: Hung-Rong Yen, Taichung City (TW); Ying-Chyi Song, Taichung City (TW); Hui-Ju Lee, Taichung City (TW); En-Ting Wu, Taichung City (TW)

(73) Assignee: China Medical University, Taichung City (TW)

(21) Appl. No.: 18/830,826

(22) Filed: Sep. 11, 2024

Related U.S. Application Data
(60) Provisional application No. 63/538,567, filed on Sep. 15, 2023.

(51) Int. Cl. G01N 33/50; G01N 33/68

(52) U.S. Cl. CPC G01N 33/5023; G01N 33/5047; G01N 33/6863; G01N 2333/415; G01N 2400/50

(57) ABSTRACT

An immunity detecting kit includes an immune-stimulant agent and a Chinese herb combination or a nutrient combination. The immune-stimulant agent includes a phorbol 12-myristate 13-acetate (PMA) and a lipopolysaccharide (LPS). The Chinese herb combination includes at least one Chinese herb extract. The nutrient combination includes at least one nutritional product. A using method of immunity detecting kit is also provided.

### RELATED APPLICATIONS

This application claims priority to U.S. Provisional Application Ser. No. 63/538,567, filed Sep. 15, 2023, which is herein incorporated by reference.

### BACKGROUND

### Technical Field

The present disclosure relates to a detecting kit and a using method thereof. More particularly, the present disclosure relates to an immunity detecting kit for detecting an immune activity of the Chinese herb combination or the nutrient combination for an organism and a using method thereof.

### Description of Related Art

The immune system is a defense mechanism for protecting the organism against the invasion by foreign substances and removing the cancer cells from the organism. If the regulation of the immune system is disordered, it will seriously impact on human health. For example, when the immune system is over reactive, the autoimmune diseases, the inflammation and the allergic reaction may occur, and when the immune system is weaker, it is insufficient to defend against the infection and to remove the cancer cells.

The traditional Chinese medicine has a unique system of treatment. The traditional Chinese medicine uses Chinese herbal medicines, nutritional products, Chinese patent medicines, diet therapy and other methods to diagnose and treat so as to regulate the immunity and achieve the aims of disease treatment. As shown in the current researching results, Angelicae Sinensis Radix (Danggui), Ginseng Radix et Rhizoma (Renshen) and other Chinese herbal medicines having the effect of benefiting qi and nourishing blood can be used to promote the macrophages and the dendritic cells to secrete TNF-α, IFN-γ, IL-2 or other cytokines to enhance the immune response, and Houttuyniae Herba (Yuxingcao), Isatidis Radix (Banlangen) and other Chinese herbal medicines having the effect of clearing heat and promoting diuresis can be used to suppress the secretion of the inflammation-related cytokines such as TNF-α, IL-6, IL-1B, etc., so as to suppress the immune response.

However, the experimental conditions and the drug treatment times of the aforementioned studies are inconsistent, so it is hard to clearly understand the effects of different Chinese herbal medicines or nutritional products on the immune response of the blood cells of various individuals, and it is impossible to assess whether the immune status of the individual is suitable for enhancing or suppressing. Further, the degrees of immune responses in response to different Chinese herbal medicines or nutritional products are also different. Currently, there is no effective method to detect the impact on the immune system of individuals after taking Chinese herbal medicines or nutritional products.

Therefore, how to provide a kit to detect the impact of Chinese herbal medicines or nutritional products on the immunity of an individual so as to assess the immune activity thereof and facilitate the formulation of the treatment strategy has become a goal pursued by the relevant field.

### SUMMARY

According to one aspect of the present disclosure, an immunity detecting kit includes an immune-stimulant agent and a Chinese herb combination. The immune-stimulant agent includes a phorbol 12-myristate 13-acetate (PMA) and a lipopolysaccharide (LPS). The Chinese herb combination includes at least one Chinese herb extract, and the at least one Chinese herb extract includes a Scutellariae Radix (Huangqin) extract, a Massa Medicata Fermentata (Shenqu) extract, a Spreading Hedyotis Herb (Baihuasheshecao) extract, a Scutellariae Barbatae Herba (Banzhilian) extract, a Schisandrae Chinensis Fructus (Wuweizi) extract, a Saposhnikoviae Radix (Fangfeng) extract, a Dioscoreae Rhizoma (Shanyao) extract, a Reynoutriae Multiflorae Radix (Heshouwu) extract, a Polygonati Rhizoma (Huangjing) extract, a Polyporus (Zhuling) extract, an Achyranthis Bidentatae Radix (Niuxi) extract, a Lycii Fructus (Gouqizi) extract, a Jujubae Fructus (Dazao) extract, a Salviae Miltiorrhizae Radix et Rhizoma (Danshen) extract, a Panacis Quinquefolii Radix (Xiyangshen) extract, a Ginseng Radix et Rhizoma (Renshen) extract, a Curcumae Longae Rhizoma (Jianghuang) extract, a Zingiberis Rhizoma (Ganjiang) extract, a Cinnamomi Cortex (cinnamon bark) extract, an Astragali Radix (Huangqi) extract, a Rehmanniae Radix Praeparata (Shudihuang) extract, a Paeoniae Radix Alba (Baishao) extract, a Chuanxiong Rhizoma (Chuanxiong) extract, an Angelicae Sinensis Radix (Danggui) extract, a Glycyrrhizae Radix et Rhizoma (Gancao) extract, a Poria (Fuling) extract, an Atractylodis Macrocephalae Rhizoma (Baizhu) extract, a Codonopsis Radix (Dangshen) extract, a Cyathulae Radix (Chuanniuxi) extract, an Anemarrhenae Rhizoma (Zhimu) extract, a Moutan Radicis Cortex (Mudanpi) extract, a Lycii Radicis Cortex (Digupi) extract, a Siegesbeckiae Herba (Xixiancao) extract, a Taxilli Herba (Sangjisheng) extract, a Gentianae Macrophyllae Radix (Qinjiao) extract, an Artemisiae Scopariae Herba (Yinchen) extract, a Reynoutriae Rhizoma et Radix (Huzhang) extract, a Plantaginis Semen (Checianzi) extract, a Taraxaci Herba (Pugongying) extract, a Lonicerae Japonicae Flos (Jinyinhua) extract, a Houttuyniae Herba (Yuxingcao) extract, an Isatidis Radix (Banlangen) extract, a Rhei Radix et Rhizoma (Dahuang) extract, a Cassiae Semen (Juemingzi) extract, a Phellodendri Cortex (Huangbo) extract, an Isatidis Folium (Daqingye) extract, a Gentianae Radix et Rhizoma (Longdan) extract, a Forsythiae Fructus (Lianqiao) extract, a Coptidis Rhizoma (Huanglian) extract, a Gardeniae Fructus (Zhizi) extract, a Tremella fuciformis extract, a Trachelospermi Caulis cum Folium (Luoshiteng) extract, a Ganoderma (Lingzhi) extract, an Antrodia cinnamomea extract and a Cordyceps (Dongchongxiacao) extract.

According to another aspect of the present disclosure, a using method of immunity detecting kit includes following steps. A sample processing step is performed, wherein a blood sample is preprocessed so as to obtain a blood-cell isolate, and the blood-cell isolate includes a plurality of immune cells. The immunity detecting kit according to the aforementioned aspect is provided. A culturing step is performed, wherein a part of the plurality of immune cells is treated with the immune-stimulant agent for a first culturing time so as to obtain a first culturing mixture, and another part of the plurality of immune cells is treated with the immune-stimulant agent and the Chinese herb combination for a second culturing time so as to obtain a second culturing mixture. A detecting step is performed, wherein an expressed amount of cytokine of the first culturing mixture is analyzed so as to obtain a first result, and an expressed amount of cytokine of the second culturing mixture is analyzed so as to obtain a second result. An analyzing step is performed, wherein the first result is compared with the second result so as to assess an immune activity of the Chinese herb combination for the plurality of immune cells. When the second result is larger than or equal to the first result, the Chinese herb combination has an immunity enhancing activity for the plurality of immune cells. When the second result is smaller than the first result, the Chinese herb combination has an immunity suppressing activity for the plurality of immune cells.

According to further another aspect of the present disclosure, an immunity detecting kit includes an immune-stimulant agent and a nutrient combination. The immune-stimulant agent includes a phorbol 12-myristate 13-acetate and a lipopolysaccharide. The nutrient combination includes at least one nutritional product, and the at least one nutritional product includes a chicken broth concentrate, a clam essence, a cyanobacteria extract, a soy isoflavone, a cranberry extract, a catechin, a Grifola frondosa extract, a fucoidan, a vitamin B and a vitamin C.

According to still another aspect of the present disclosure, a using method of immunity detecting kit includes following steps. A sample processing step is performed, wherein a blood sample is preprocessed so as to obtain a blood-cell isolate, and the blood-cell isolate includes a plurality of immune cells. The immunity detecting kit according to the aforementioned aspect is provided. A culturing step is performed, wherein a part of the plurality of immune cells is treated with the immune-stimulant agent for a first culturing time so as to obtain a first culturing mixture, and another part of the plurality of immune cells is treated with the immune-stimulant agent and the nutrient combination for a second culturing time so as to obtain a second culturing mixture. A detecting step is performed, wherein an expressed amount of cytokine of the first culturing mixture is analyzed so as to obtain a first result, and an expressed amount of cytokine of the second culturing mixture is analyzed so as to obtain a second result. An analyzing step is performed, wherein the first result is compared with the second result so as to assess an immune activity of the nutrient combination for the plurality of immune cells. When the second result is larger than or equal to the first result, the nutrient combination has an immunity enhancing activity for the plurality of immune cells. When the second result is smaller than the first result, the nutrient combination has an immunity suppressing activity for the plurality of immune cells.

### BRIEF DESCRIPTION OF THE DRAWINGS

The present disclosure can be more fully understood by reading the following detailed description of the embodiment, with reference made to the accompanying drawings as follows:

FIG. 1 is a flow chart of a using method of immunity detecting kit according to one embodiment of the present disclosure.

FIG. 2 is a flow chart of a using method of immunity detecting kit according to another embodiment of the present disclosure.

FIG. 3 shows the results of the immunity values of different groups of subjects.

FIG. 4A shows the results of the immunity value of breast cancer patients after accepting the screened Chinese herbal extracts.

FIG. 4B shows the results of the immunity value of endometrial cancer patients after accepting the screened Chinese herbal extracts.

FIG. 4C shows the results of the immunity value of ovarian cancer patients after accepting the screened Chinese herbal extracts.

FIG. 4D shows the results of the immunity value of colorectal cancer patients after accepting the screened Chinese herbal extracts.

FIG. 5A shows the proportion distribution of the breast cancer patients with different body constitutions in traditional Chinese medicine.

FIG. 5B shows the proportion distribution of the endometrial cancer patients with different body constitutions in traditional Chinese medicine.

FIG. 5C shows the proportion distribution of the ovarian cancer patients with different body constitutions in traditional Chinese medicine.

FIG. 5D shows the proportion distribution of the colorectal cancer patients with different body constitutions in traditional Chinese medicine.

FIG. 6A shows the results of the immunity value of the breast cancer patients with Gentleness constitution after accepting the screened Chinese herbal extracts.

FIG. 6B shows the results of the immunity value of the breast cancer patients with Qi-deficiency constitution after accepting the screened Chinese herbal extracts.

FIG. 6C shows the results of the immunity value of the breast cancer patients with Phlegm-dampness constitution after accepting the screened Chinese herbal extracts.

FIG. 6D shows the results of the immunity value of the breast cancer patients with Qi-depression constitution after accepting the screened Chinese herbal extracts.

FIG. 6E shows the results of the immunity value of the breast cancer patients with Blood-stasis constitution after accepting the screened Chinese herbal extracts.

FIG. 6F shows the results of the immunity value of the breast cancer patients with Yin-deficiency constitution after accepting the screened Chinese herbal extracts.

FIG. 6G shows the results of the immunity value of the breast cancer patients with Yang-deficiency constitution after accepting the screened Chinese herbal extracts.

FIG. 6H shows the results of the immunity value of the breast cancer patients with Dampness-heat constitution after accepting the screened Chinese herbal extracts.

FIG. 61 shows the results of the immunity value of the breast cancer patients with Special diathesis constitution after accepting the screened Chinese herbal extracts.

FIG. 7A shows the results of the immunity value of the breast cancer patient of Example 1 in response to different Chinese herbal extracts.

FIG. 7B shows the results of the immunity value of the breast cancer patient of Example 1 after accepting the Schisandrae Chinensis Fructus extract for two weeks.

FIG. 8A shows the expressed amounts of IL-6 of the non-hypertensive patients and the hypertensive patients.

FIG. 8B shows the sum of the expressed amounts of IL-6, TNF-α and IL-1B of the non-hypertensive patients and the hypertensive patients.

FIG. 9 shows the results of the immunity value of the hypertensive patient of Example 2 in response to different Chinese herbal extracts.

FIG. 10A shows the average expressed amounts of IFN-γ, TNF-α and IL-2 of the health subjects in response to different Chinese herbal extracts.

FIG. 10B shows the average expressed amounts of IFN-γ, TNF-α and IL-2 of the health subjects in response to different Chinese herbal extracts and nutritional products.

FIG. 11A shows the sum of the average expressed amounts of IFN-γ, TNF-α and IL-2 of the health subjects in response to different Chinese herbal extracts.

FIG. 11B shows the sum of the average expressed amounts of IFN-γ, TNF-α and IL-2 of the health subjects in response to different Chinese herbal extracts and nutritional products.

### DETAILED DESCRIPTION

The present disclosure will be further exemplified by the following specific embodiments to facilitate utilizing and practicing the present disclosure completely by the people skilled in the art without over-interpreting and over-experimenting. However, these practical details are used to describe how to implement the materials and methods of the present disclosure and are not necessary.

### [The Immunity Detecting Kit of the Present Disclosure]

According to one embodiment of the present disclosure, the immunity detecting kit includes an immune-stimulant agent and a Chinese herb combination. The immune-stimulant agent includes a phorbol 12-myristate 13-acetate (PMA) and a lipopolysaccharide (LPS). The Chinese herb combination includes at least one Chinese herb extract, and the at least one Chinese herb extract includes a Scutellariae Radix (Huangqin) extract, a Massa Medicata Fermentata (Shenqu) extract, a Spreading Hedyotis Herb (Baihuasheshecao) extract, a Scutellariae Barbatae Herba (Banzhilian) extract, a Schisandrae Chinensis Fructus (Wuweizi) extract, a Saposhnikoviae Radix (Fangfeng) extract, a Dioscoreae Rhizoma (Shanyao) extract, a Reynoutriae Multiflorae Radix (Heshouwu) extract, a Polygonati Rhizoma (Huangjing) extract, a Polyporus (Zhuling) extract, an Achyranthis Bidentatae Radix (Niuxi) extract, a Lycii Fructus (Gouqizi) extract, a Jujubae Fructus (Dazao) extract, a Salviae Miltiorrhizae Radix et Rhizoma (Danshen) extract, a Panacis Quinquefolii Radix (Xiyangshen) extract, a Ginseng Radix et Rhizoma (Renshen) extract, a Curcumae Longae Rhizoma (Jianghuang) extract, a Zingiberis Rhizoma (Ganjiang) extract, a Cinnamomi Cortex (cinnamon bark) extract, an Astragali Radix (Huangqi) extract, a Rehmanniae Radix Praeparata (Shudihuang) extract, a Paeoniae Radix Alba (Baishao) extract, a Chuanxiong Rhizoma (Chuanxiong) extract, an Angelicae Sinensis Radix (Danggui) extract, a Glycyrrhizae Radix et Rhizoma (Gancao) extract, a Poria (Fuling)) extract, an Atractylodis Macrocephalae Rhizoma (Baizhu) extract, a Codonopsis Radix (Dangshen) extract, a Cyathulae Radix (Chuanniuxi) extract, an Anemarrhenae Rhizoma (Zhimu) extract, a Moutan Radicis Cortex (Mudanpi) extract, a Lycii Radicis Cortex (Digupi) extract, a Siegesbeckiae Herba (Xixiancao) extract, a Taxilli Herba (Sangjisheng) extract, a Gentianae Macrophyllae Radix (Qinjiao) extract, an Artemisiae Scopariae Herba (Yinchen) extract, a Reynoutriae Rhizoma et Radix (Huzhang) extract, a Plantaginis Semen (Checianzi) extract, a Taraxaci Herba (Pugongying) extract, a Lonicerae Japonicae Flos (Jinyinhua) extract, a Houttuyniae Herba (Yuxingcao) extract, an Isatidis Radix (Banlangen) extract, a Rhei Radix et Rhizoma (Dahuang) extract, a Cassiae Semen (Juemingzi) extract, a Phellodendri Cortex (Huangbo) extract, an Isatidis Folium (Daqingye) extract, a Gentianae Radix et Rhizoma (Longdan) extract, a Forsythiae Fructus (Lianqiao) extract, a Coptidis Rhizoma (Huanglian) extract, a Gardeniae Fructus (Zhizi) extract, a Tremella fuciformis extract, a Trachelospermi Caulis cum Folium (Luoshiteng) extract, a Ganoderma (Lingzhi) extract, an Antrodia cinnamomea extract and a Cordyceps (Dongchongxiacao) extract.

In detail, by the arrangement that the immune-stimulant agent includes the phorbol 12-myristate 13-acetate and the lipopolysaccharide, the immune-stimulant agent of the immunity detecting kit of the present disclosure can effectively induce the expression of cytokines. In particular, the phorbol 12-myristate 13-acetate is a phorbol ester for inducing the activity of the protein kinase C (PKC) so as to activate the NF-KB or other transcription factors and then to promote the expressions of the proteins related to T-cells and the monocyte in blood. The lipopolysaccharide is the main component of the outer membrane of Gram-negative bacteria and can be used to stimulate the immune cells to secrete IL-6, TNF-α or other cytokines and then to activate the inflammatory response. Therefore, the immune-stimulant agent can be used to activate the immune cells and then facilitate the interaction of the Chinese herb combination and the immune cells, and it is favorable for assessing the immune activity of the Chinese herb combination for the organism.

In the immunity detecting kit of the present disclosure, the fifty-five kinds of Chinese herbal extracts of the Chinese herb combination are common single herbs and are generally used in medicinal diets, dietary supplements and spices, wherein the Phellodendri Cortex extract can include the extract of Phellodron chinense Schneid., and the Poria extract can include the extract of the core portion thereof.

Further, in the immunity detecting kit of the present disclosure, the Chinese herb combination can further include a Long-Dan-Xie-Gan-Tang, a Yin-Chen-Hao-Tang, a Ban-Xia-Xie-Xin-Tang, a San-Huang-Xie-Xin-Tang, a Si-Jun-Zi-Tang, a Si-Wu-Tang, a Shi-Quan-Da-Bu-Tang, a Bu-Zhong-Yi-Qi-Tang, a Sheng-Mai-San, a Shen-Ling-Bai-Zhu-San, a Sheng-Yu-Tang, a Gui-Pi-Tang, a Ren-Shen-Yang-Rong-Tang, a Liu-Wei-Di-Huang-Wan, a Yu-Ping-Feng-San, a Jia-Wei-Xiao-Yao-San, a Ping-Wei-San, a Siang-Sha-Liu-Jun-Zi-Tang, a Zhi-Bo-Di-Huang-Wan and a You-Gui-Wan.

In detail, the Long-Dan-Xie-Gan-Tang, the Yin-Chen-Hao-Tang, Ban-Xia-Xie-Xin-Tang, the San-Huang-Xie-Xin-Tang, the Si-Jun-Zi-Tang, the Si-Wu-Tang, the Shi-Quan-Da-Bu-Tang, the Bu-Zhong-Yi-Qi-Tang, the Sheng-Mai-San, the Shen-Ling-Bai-Zhu-San, the Sheng-Yu-Tang, the Gui-Pi-Tang, the Ren-Shen-Yang-Rong-Tang, the Liu-Wei-Di-Huang-Wan, the u-Ping-Feng-San, the Jia-Wei-Xiao-Yao-San, the Ping-Wei-San, the Siang-Sha-Liu-Jun-Zi-Tang, the Zhi-Bo-Di-Huang-Wan and the You-Gui-Wan are common Chinese herbal formulas and can be used to relieve or treat different diseases of an individual by various compositions of traditional Chinese medicine. The Long-Dan-Xie-Gan-Tang includes Gentianae Radix et Rhizoma, Scutellariae Radix, Gardeniae Fructus, Alismatis Rhizoma (Zexie), Akebiae Caulis (Mutong), Plantaginis Semen, Angelicae Sinensis Radix, Rehmanniae Radix (Shengdihuang), Bupleuri Radix (Chaihu), Glycyrrhizae Radix et Rhizoma and other Chinese herbal medicines. The Yin-Chen-Hao-Tang includes Artemisiae Scopariae Herba, Rhei Radix et Rhizoma, Gardeniae Fructus and other Chinese herbal medicines. The Ban-Xia-Xie-Xin-Tang includes Pinelliae Rhizoma Praeparatum (Zhibanxia), Scutellariae Radix, Zingiberis Rhizoma, Ginseng Radix et Rhizoma, Coptidis Rhizoma, Jujubae Fructus, Glycyrrhizae Radix et Rhizoma Praeparata Cum Melle (Zhigancao) and other Chinese herbal medicines. The San-Huang-Xie-Xin-Tang includes Rhei Radix et Rhizoma, Coptidis Rhizoma, Scutellariae Radix and other Chinese herbal medicines. The Si-Jun-Zi-Tang includes Ginseng Radix et Rhizoma, Poria, Glycyrrhizae Radix et Rhizoma Praeparata Cum Melle, Atractylodis Macrocephalae Rhizoma, Zingiberis Rhizoma Recens (Shengjiang), Jujubae Fructus and other Chinese herbal medicines. The Si-Wu-Tang includes Rehmanniae Radix Praeparata, Paeoniae Radix Alba, Angelicae Sinensis Radix, Chuanxiong Rhizoma and other Chinese herbal medicines. The Shi-Quan-Da-Bu-Tang includes Poria, Atractylodis Macrocephalae Rhizoma, Ginseng Radix et Rhizoma, Rehmanniae Radix Praeparata, Paeoniae Radix Alba, Glycyrrhizae Radix et Rhizoma Praeparata Cum Melle, Astragali Radix, Cinnamomi Cortex, Angelicae Sinensis Radix, Chuanxiong Rhizoma and other Chinese herbal medicines. The Bu-Zhong-Yi-Qi-Tang includes Astragali Radix, Ginseng Radix et Rhizoma, Atractylodis Macrocephalae Rhizoma, Glycyrrhizae Radix et Rhizoma Praeparata Cum Melle, Angelicae Sinensis Radix, Citri Reticulatae Pericarpium Vetum (Chenpi), Cimicifugae Rhizoma (Shengma), Bupleuri Radix, Zingiberis Rhizoma Recens, Jujubae Fructus and other Chinese herbal medicines. Sheng-Mai-San includes Ginseng Radix et Rhizoma, Ophiopogonis Radix (Maimendong), Schisandrae Chinensis Fructus and other Chinese herbal medicines. The Shen-Ling-Bai-Zhu-San includes Lablab Semen Album (Baibiandou), Ginseng Radix et Rhizoma, Poria, Atractylodis Macrocephalae Rhizoma, Glycyrrhizae Radix et Rhizoma, Dioscoreae Rhizoma, Nelumbinis Semen (Lianzi), Platycodonis Radix (Jiegeng), Coicis Semen (Yiyiren), Amomi Fructus (Sharen) and other Chinese herbal medicines. The Sheng-Yu-Tang includes Rehmanniae Radix Praeparata, Chuanxiong Rhizoma, Ginseng Radix et Rhizoma, Angelicae Sinensis Radix, Astragali Radix, Paeoniae Radix Alba and other Chinese herbal medicines. The Gui-Pi-Tang includes Ginseng Radix et Rhizoma, Longan Arillus (Longyanrou), Astragali Radix, Glycyrrhizae Radix et Rhizoma Praeparata Cum Melle, Atractylodis Macrocephalae Rhizoma, Poria, Aucklandiae Radix (Muxiang), Angelicae Sinensis Radix, Ziziphi Spinosae Semen (Suanzaoren), Polygalae Radix (Yuanzhi), Zingiberis Rhizoma Recens, Jujubae Fructus and other Chinese herbal medicines. The Ren-Shen-Yang-Rong-Tang includes Paeoniae Radix Alba, Angelicae Sinensis Radix, Cinnamomi Cortex Centralis (Gueisin), Glycyrrhizae Radix et Rhizoma Praeparata Cum Melle, Citri Reticulatae Pericarpium Vetum, Ginseng Radix et Rhizoma, Atractylodis Macrocephalae Rhizoma, Astragali Radix, Rehmanniae Radix Praeparata, Schisandrae Chinensis Fructus, Poria, Polygalae Radix, Zingiberis Rhizoma Recens, Jujubae Fructus and other Chinese herbal medicines. The Liu-Wei-Di-Huang-Wan includes Rehmanniae Radix Praeparata, Corni Sarcocarpium (Shanzhuyu), Dioscoreae Rhizoma, Alismatis Rhizoma, Moutan Radicis Cortex, Poria and other Chinese herbal medicines. The Yu-Ping-Feng-San includes Astragali Radix, Saposhnikoviae Radix, Atractylodis Macrocephalae Rhizoma and other Chinese herbal medicines. The Jia-Wei-Xiao-Yao-San includes Angelicae Sinensis Radix, Atractylodis Macrocephalae Rhizoma, Paeoniae Radix Alba, Bupleuri Radix, Poria, Glycyrrhizae Radix et Rhizoma Praeparata Cum Melle, Moutan Radicis Cortex, Gardeniae Fructus, Zingiberis Rhizoma Tostum, Menthae Herba (Bohe) and other Chinese herbal medicines. The Ping-Wei-San includes Citri Reticulatae Pericarpium Vetum, Magnoliae Cortex (Houpu), Glycyrrhizae Radix et Rhizoma Praeparata Cum Melle, Atractylodis Rhizoma (Cangzu), Zingiberis Rhizoma Recens, Jujubae Fructus and other Chinese herbal medicines. The Siang-Sha-Liu-Jun-Zi-Tang includes Ginseng Radix et Rhizoma, Atractylodis Macrocephalae Rhizoma, Poria, Glycyrrhizae Radix et Rhizoma, Citri Reticulatae Pericarpium Vetum, Pinelliae Rhizoma Praeparatum, Amomi Fructus, Aucklandiae Radix, Zingiberis Rhizoma Recens and other Chinese herbal medicines. The Zhi-Bo-Di-Huang-Wan includes Rehmanniae Radix Praeparata, Corni Sarcocarpium, Poria, Dioscoreae Rhizoma, Moutan Radicis Cortex, Alismatis Rhizoma, Anemarrhenae Rhizoma, Phellodendri Cortex and other Chinese herbal medicines. The You-Gui-Wan includes Rehmanniae Radix Praeparata, Dioscoreae Rhizoma, Lycii Fructus, Cuscutae Semen (Tusizi), Eucommiae Cortex (Duzhong), Deerhorn glue, Corni Sarcocarpium, Angelicae Sinensis Radix, Aconiti Lateralis Radix Praeparata (Zifuzi), Cinnamomi Cortex and other Chinese herbal medicines.

Further, in the immunity detecting kit of the present disclosure, the Chinese herb extracts of the Chinese herb combination can be prepared by water extraction method, and the Chinese herb extract can be sterilized by high pressure and stored at −80° C. so as to prevent the microorganisms from growing and prevent the deterioration of the Chinese herb combination and then destroying the activity thereof. Hence, it is favorable for enhancing the detecting efficiency of the immunity detecting kit of the present disclosure, but the present disclosure is not limited thereof.

Further, the immunity detecting kit of the present disclosure can further include an endotoxin inhibitor, and the endotoxin inhibitor can include a polymyxin B (PMB). The endotoxin is a natural compound derived from bacteria and is widely found in nature. The endotoxin can induce the inflammatory responses of the organism so as to promote the secretion of cytokines by the immune cells. Therefore, by the arrangement that the immunity detecting kit includes the endotoxin inhibitor, the endotoxin inhibitor can inhibit the inflammatory response caused by the endotoxin, and it is favorable for ensuring that the activation of the immune cells is only caused by the immune-stimulant agent and the Chinese herb combination. Thus, it is favorable for enhancing the detecting efficiency of the immunity detecting kit of the present disclosure.

Further, in the immunity detecting kit of the present disclosure, a concentration of the immune-stimulant agent can be 50 ng/ml to 200 ng/ml, a concentration of the Chinese herb combination can be 50 μg/mL to 200 μg/mL, and a concentration of the endotoxin inhibitor can be 10 μg/mL to 60 μg/mL.

Therefore, by the arrangement that the immunity detecting kit includes the immune-stimulant agent and the Chinese herb combination, the immunity detecting kit of the present disclosure can be applied to assess the immune activity of the Chinese herb combination for an individual so as to screen a Chinese herb extract with an immunity enhancing activity or an immunity suppressing activity. Thus, it is favorable for screening the Chinese herbal extracts suitable for the current immune status of the individual based on different needs so as to facilitate the formulation of the treatment strategy.

According to another embodiment of the present disclosure, the immunity detecting kit includes an immune-stimulant agent and a nutrient combination. The immune-stimulant agent includes a phorbol 12-myristate 13-acetate and a lipopolysaccharide. The nutrient combination includes at least one nutritional product, and the at least one nutritional product includes a chicken broth concentrate, a clam essence, a cyanobacteria extract, a soy isoflavone, a cranberry extract, a catechin, a Grifola frondosa extract, a fucoidan, a vitamin B and a vitamin C. The details of the phorbol 12-myristate 13-acetate and the lipopolysaccharide are the same as those shown in the aforementioned description and will not be described herein. Further, the aforementioned 10 kinds of nutritional products are the common supplementary food in the market and include the active ingredients of plants or animals, and thus they are widely used as supplements to prevent disease or maintain health.

Further, the immunity detecting kit of the present disclosure can further include an endotoxin inhibitor, and the endotoxin inhibitor can include a polymyxin B. Therefore, by the arrangement that the immunity detecting kit includes the endotoxin inhibitor, the endotoxin inhibitor can inhibit the inflammatory response caused by the endotoxin, and it is favorable for ensuring that the activation of the immune cells is only caused by the immune-stimulant agent and the nutrient combination. Thus, it is favorable for enhancing the detecting efficiency of the immunity detecting kit of the present disclosure.

Further, in the immunity detecting kit of the present disclosure, a concentration of the immune-stimulant agent can be 50 ng/ml to 200 ng/ml, a concentration of the endotoxin inhibitor can be 10 μg/mL to 60 μg/mL, and a concentration of the nutrient combination can be 50 μg/mL to 200 μg/mL.

Therefore, by the arrangement that the immunity detecting kit includes the immune-stimulant agent and the nutrient combination, the immunity detecting kit of the present disclosure can be applied to assess the immune activity of the nutrient combination for an individual so as to screen a nutrient product with an immunity enhancing activity or an immunity suppressing activity from the nutrient combination. Thus, it is favorable for providing a reference to selecting the nutritional products.

### [The Using Method of Immunity Detecting Kit of the Present Disclosure]

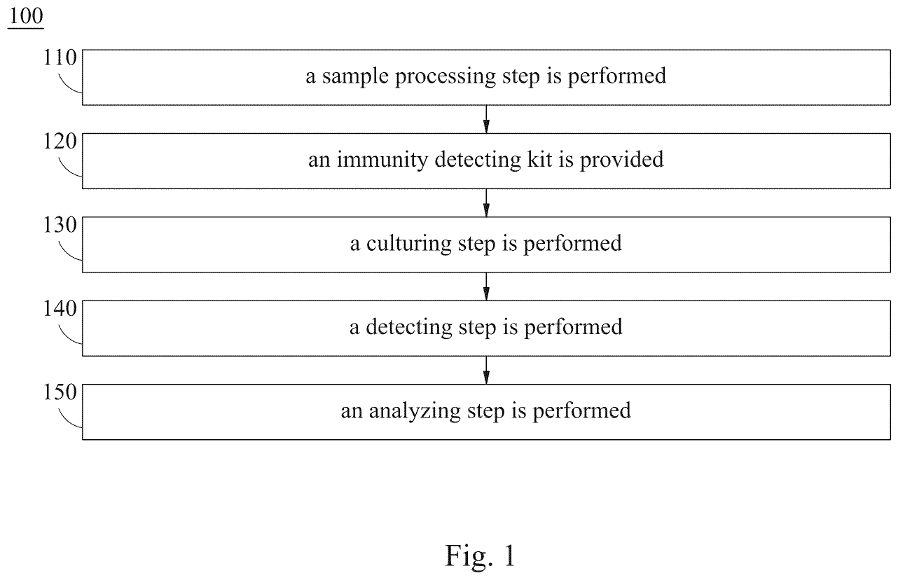

Reference is made to FIG. 1, which shows a flow chart of a using method 100 of immunity detecting kit according to one embodiment of the present disclosure. The using method 100 of immunity detecting kit includes Step 110, Step 120, Step 130, Step 140 and Step 150.

In Step 110, a sample processing step is performed, wherein a blood sample is preprocessed so as to obtain a blood-cell isolate, and the blood-cell isolate includes a plurality of immune cells. In detail, the blood sample can be stored in the vacutainer including ethylene diamine tetraacetic acid (EDTA) and then centrifuged to separate the plasma and the blood cells of the blood sample, the blood cells are collected so as to obtain the blood-cell isolate, and the immune cells of the blood-cell isolate are collected for further analysis. Further, each of the immune cells can be a monocyte.

In Step 120, an immunity detecting kit is provided. In the using method 100 of immunity detecting kit of the present disclosure, the immunity detecting kit includes an immune-stimulant agent and a Chinese herb combination, wherein the details of the immune-stimulant agent and the Chinese herb combination are described in the foregoing description and will not be described herein.

In Step 130, a culturing step is performed, wherein a part of the immune cells is treated with the immune-stimulant agent for a first culturing time so as to obtain a first culturing mixture, and another part of the immune cells is treated with the immune-stimulant agent and the Chinese herb combination for a second culturing time so as to obtain a second culturing mixture. In detail, a concentration of the immune-stimulant agent can be 50 ng/ml to 200 ng/ml, and a concentration of the Chinese herb combination can be 50 μg/mL to 200 μg/mL. Further, the first culturing time can be 24 hours to 48 hours, and the second culturing time can be 24 hours to 48 hours.

In detail, in Step 130, the total of the immune cells collected in the sample processing step are divided into two equal parts, and the immune cells of each of the parts are seeded in the 96-wells plate in a number of 2×10⁵ cells/well. Then, the immune-stimulant agent or the immune-stimulant agent along with the Chinese herb combination are added to each well and reacted for 24 hours to 48 hours under 37° C. Finally, the supernatants are collected so as to obtain the first culturing mixture and the second culturing mixture.

Further, when the immune-stimulant agent is the phorbol 12-myristate 13-acetate, one part of the immune cells is simultaneously treated with the immune-stimulant agent and the endotoxin inhibitor so as to obtain the first culturing mixture, and the other part of the immune cells is simultaneously treated with the immune-stimulant agent, the Chinese herb combination and the endotoxin inhibitor so as to obtain the second culturing mixture. Therefore, the endotoxin inhibitor can be used to reduce the impact on the activation of the immunity caused by the factors other than the phorbol 12-myristate 13-acetate and the Chinese herb combination, so the accuracy and the reliability of the following tests can be enhanced.

In Step 140, a detecting step is performed, wherein an expressed amount of cytokine of the first culturing mixture is analyzed so as to obtain a first result, and an expressed amount of cytokine of the second culturing mixture is analyzed so as to obtain a second result. In detail, the expressed amount of cytokine of the first culturing mixture and the expressed amount of cytokine of the second culturing mixture can be detected by the enzyme-linked immunosorbent assay (ELISA), wherein the cytokine of the first culturing mixture can include TNF-α, IFN-γ, IL-2, IL-6 and IL-1B, and the cytokine of the second culturing mixture can include TNF-α, IFN-γ, IL-2, IL-6 and IL-1B.

In Step 150, an analyzing step is performed, wherein the first result is compared with the second result so as to assess an immune activity of the Chinese herb combination for the plurality of immune cells. In detail, when the second result is larger than or equal to the first result, the Chinese herb combination has an immunity enhancing activity for the plurality of immune cells; and when the second result is smaller than the first result, the Chinese herb combination has an immunity suppressing activity for the plurality of immune cells. In particular, the first result represents the immune activity of the immune cells in the blood sample, and the second result represents the effects of the Chinese herb combination on the immune cells in the blood sample. Therefore, by comparing the first result and the second result, it is favorable for screening the Chinese herbal extracts of the Chinese herb combination that can be used to activate the immune response of the immune cells or suppress the immune response of the immune cells so as to facilitate the formulation of the treatment strategy.

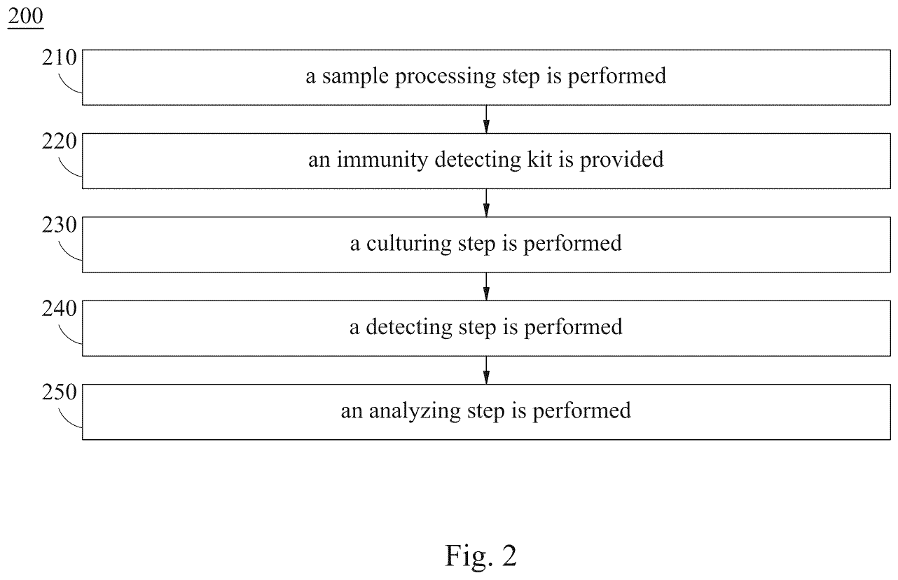

Reference is made to FIG. 2, which is a flow chart of a using method 200 of immunity detecting kit according to another embodiment of the present disclosure. The using method 200 of immunity detecting kit includes Step 210, Step 220, Step 230, Step 240 and Step 250.

In Step 210, a sample processing step is performed, wherein a blood sample is preprocessed so as to obtain a blood-cell isolate, and the blood-cell isolate includes a plurality of immune cells. The details of Step 210 are the same as those shown in Step 110 of FIG. 1 and will not be described herein.

In Step 220, an immunity detecting kit is provided. In the using method 200 of immunity detecting kit of the present disclosure, the immunity detecting kit includes an immune-stimulant agent and a nutrient combination, wherein the details of the immune-stimulant agent and the nutrient combination are described in the aforementioned description and will not be described herein.

In Step 230, a culturing step is performed, wherein a part of the immune cells is treated with the immune-stimulant agent for a first culturing time so as to obtain a first culturing mixture, and another part of the immune cells is treated with the immune-stimulant agent and the nutrient combination for a second culturing time so as to obtain a second culturing mixture. In detail, a concentration of the immune-stimulant agent can be 50 ng/mL to 200 ng/ml, and a concentration of the nutrient combination can be 50 μg/mL to 200 μg/mL. Further, the first culturing time can be 24 hours to 48 hours, and the second culturing time can be 24 hours to 48 hours.

In detail, in Step 230, the total of the immune cells collected in the sample processing step are divided into two equal parts, and the immune cells of each of the parts are seeded in the 96-wells plate in a number of 2×10⁵ cells/well. Then, the immune-stimulant agent or the immune-stimulant agent along with the nutrient combination are added to each well and reacted for 24 hours to 48 hours under 37° C. Finally, the supernatants are collected so as to obtain the first culturing mixture and the second culturing mixture.

Further, when the immune-stimulant agent is the phorbol 12-myristate 13-acetate, one part of the immune cells is simultaneously treated with the immune-stimulant agent and the endotoxin inhibitor so as to obtain the first culturing mixture, and the other part of the immune cells is simultaneously treated with the immune-stimulant agent, the nutrient combination and the endotoxin inhibitor so as to obtain the second culturing mixture. Therefore, the endotoxin inhibitor can be used to reduce the impact on the activation of the immunity caused by the factors other than the phorbol 12-myristate 13-acetate and the nutrient combination, so the accuracy and the reliability of the following tests can be enhanced.

In Step 240, a detecting step is performed, wherein an expressed amount of cytokine of the first culturing mixture is analyzed so as to obtain a first result, and an expressed amount of cytokine of the second culturing mixture is analyzed so as to obtain a second result. In detail, the expressed amount of cytokine of the first culturing mixture and the expressed amount of cytokine of the second culturing mixture can be detected by the enzyme-linked immunosorbent assay, wherein the cytokine of the first culturing mixture can include TNF-α, IFN-γ, IL-2, IL-6 and IL-1β, and the cytokine of the second culturing mixture can include TNF-α, IFN-γ, IL-2, IL-6 and IL-1β.

In Step 250, an analyzing step is performed, wherein the first result is compared with the second result so as to assess an immune activity of the nutrient combination to the plurality of immune cells. In detail, when the second result is larger than or equal to the first result, the nutrient combination has an immunity enhancing activity for the plurality of immune cells; and when the second result is smaller than the first result, the nutrient combination has an immunity suppressing activity for the plurality of immune cells. In particular, the first result represents the immune activity of the immune cells in the blood sample, and the second result represents the effects of the nutrient combination on the immune cells in the blood sample. Therefore, by comparing the first result and the second result, it is favorable for screening the nutritional products of the nutrient combination that can be used to activate the immune response of the immune cells or suppress the immune response of the immune cells so as to facilitate the formulation of the treatment strategy.

Further, the operating details of the enzyme-linked immunosorbent assay are common knowledge in the field, and the details can be adjusted according to experimental needs, so that the details will not be described herein.

### Example

The present disclosure will be further exemplified by the following specific embodiments with the drawings so as to facilitate utilizing and practicing the present disclosure completely by the people skilled in the art without over-interpreting and over-experimenting. However, the readers should understand that the present disclosure should not be limited to these practical details thereof, that is, in some embodiments, these practical details are used to describe how to implement the materials and methods of the present disclosure and are not necessary.

I. Analyzing the immune status of the health subjects and the patients suffering from different diseases

In the present experiment, the blood samples of the health subjects and the patients suffering from different diseases are collected and then analyzed according to the using method 100 of immunity detecting kit of the present disclosure so as to compare the first results of the blood samples of the health subjects with the first results of the blood samples of the patients suffering from different diseases, and then the immune status of the health subjects and the patients suffering from different diseases can be assessed.

In detail, there are blood samples of 3226 subjects are analyzed in the present experiment, and the blood samples are divided into six groups according to the physical condition of the subjects, wherein Group 1 includes 1065 blood samples of the health subjects, Group 2 includes 294 blood samples of the patients suffering from cancer, Group 3 includes 406 blood samples of the patients suffering from high blood fat, high cholesterol, high blood pressure or high blood sugar, Group 4 includes 108 blood samples of the patients suffering from hepatitis, Group 5 includes 551 blood samples of the patients suffering from ulcers, and Group 6 includes 802 blood samples of the patients suffering from other diseases. Further, the patients suffering from cancer include the subjects who had suffered from cancer before, the patients suffering from hepatitis include the patients suffering from Hepatitis B, hepatitis C, autoimmune hepatitis, cirrhosis or alcoholic hepatitis, the patients suffering from ulcers include the patients suffering from peptic ulcer, duodenal ulcer, esophageal ulcer, gastric ulcer or infected by Helicobacter pylori, and the patients suffering from other diseases include the patients suffering from allergy, goiter, gastroesophageal reflux or other diseases.

In the present experiment, the immune-stimulant agent of the immunity detecting kit is the phorbol 12-myristate 13-acetate, and the immunity detecting kit further includes the endotoxin inhibitor, wherein the concentration of the phorbol 12-myristate 13-acetate is 50 ng/ml, the endotoxin inhibitor is the polymyxin B with the concentration of 50 μg/mL, the first culturing time is 24 hours or 48 hours, and the cytokine of the first culturing mixture includes TNF-α, IFN-γ and IL-2. In detail, the inducing times of the immune response of the immune cells of the blood samples of different subjects after stimulation are different, and thus the immune cells of each of the subjects are treated with the immune-stimulant agent for 24 hours and 48 hours so as to ensure the activation of the immune response of the immune cells in the present experiment. Then, the expressed amounts of cytokine of the first culturing mixtures obtained at 24 hours and 48 hours are detected, if the immune cells have responded to the stimulation at 24 hours, the first result obtained at 24 hours is used. Alternatively, if the immune cells have not responded to the stimulation at 24 hours, the first result obtained at 48 hours is used for the following analysis.

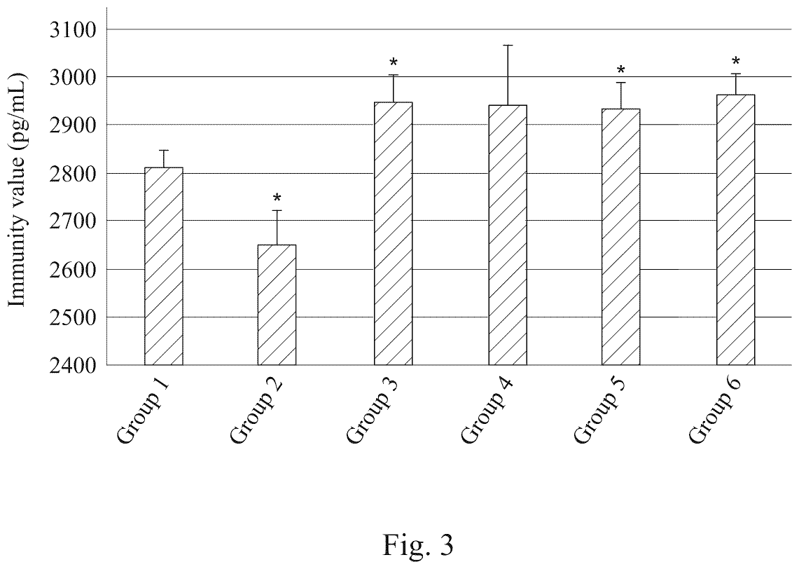

Reference is made to FIG. 3, which shows the results of the immunity values of different groups of subjects. In FIG. 3, the “immunity value” represents the sum of the expressed amounts of TNF-α, IFN-γ and IL-2, and the mark “*” represents that the data is statistically significant compared with Group 1 (p<0.05). Unless otherwise specified, if the definitions of the marks in the following figures are the same as those shown in FIG. 3, it will not be described again.

As shown in FIG. 3, compared with Group 1, the immunity value of Group 2 is significantly lower, and the immunity values of Group 3, Group 5 and Group 6 are significantly higher. It represents that the immune ability of the patients suffering from cancer is lower than that of the health subjects, and the immune responses of the patients suffering high blood fat, high cholesterol, high blood pressure or high blood sugar, the patients suffering from ulcers and the patients suffering from other diseases are stronger than that of the health subjects.

1. Analysis of the Immunity of the Patients Suffering from Cancer

In the present experiment, the first results and the second results of the 218 blood samples of the patients suffering from cancer in Group 2 are analyzed so as to compare the first result with the second result, respectively, and the Chinese herbal extracts of the Chinese herb combination having the immunity enhancing activity for the patients suffering from cancer can be screened. The screened Chinese herbal extracts suitable for different patients suffering from cancer are respectively applied thereto for two weeks, and the expressed amounts of the TNF-α, IFN-γ and IL-2 are detected. Further, the patients suffering from cancer include 138 breast cancer patients, 35 endometrial cancer patients, 26 ovarian cancer patients and 19 colorectal cancer patients. Furthermore, in the present experiment, the concentration of each of the screened Chinese herbal extracts of the Chinese herb combination is 100 μg/mL, the second culturing time is 24 hours or 48 hours, and the cytokine of the second culturing mixture includes TNF-α, IFN-γ and IL-2. Moreover, the first culturing time and the second culturing time of each of the subjects are the same so as to obtain a reliable analytical result, and the definition of the second culturing time is the same as those shown in the first culturing time and will not be described herein.

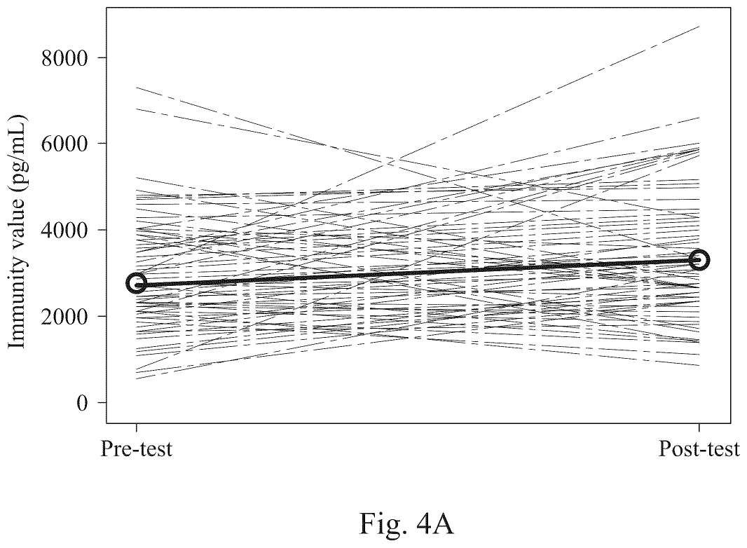

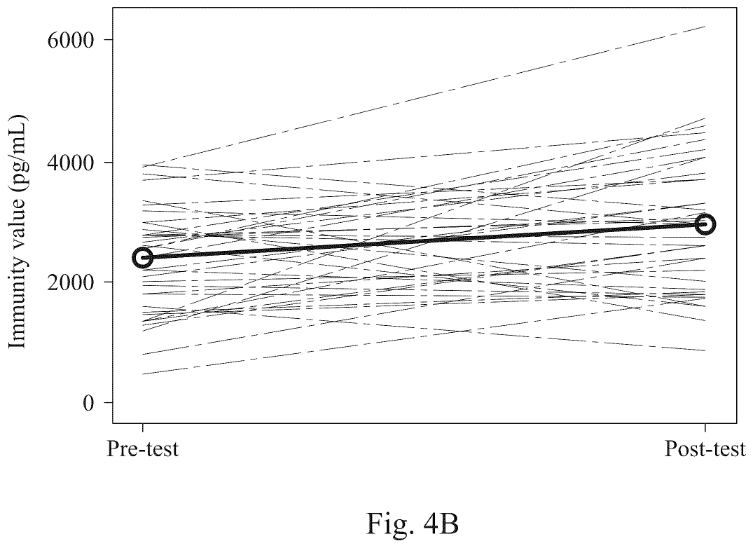

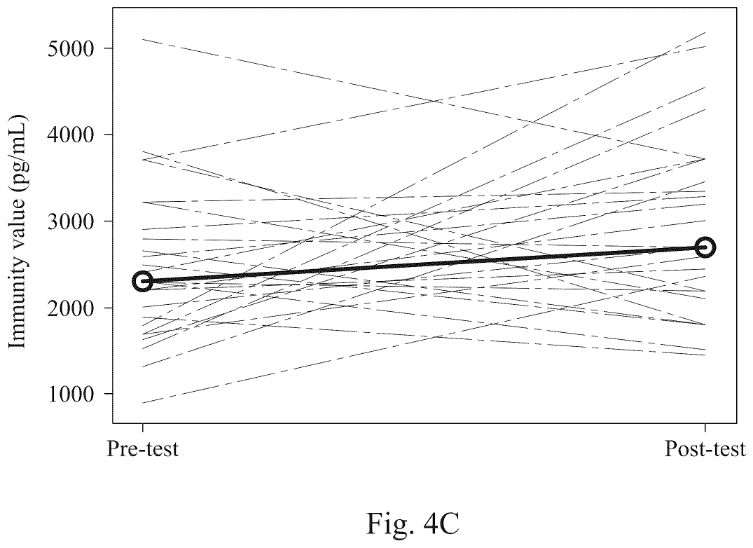

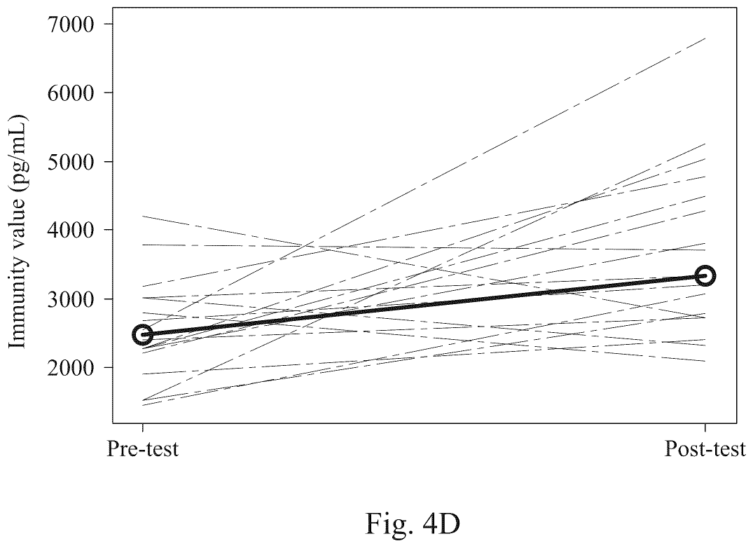

Reference is made to FIG. 4A to FIG. 4D. FIG. 4A shows the results of the immunity value of breast cancer patients after accepting the screened Chinese herbal extracts. FIG. 4B shows the results of the immunity value of endometrial cancer patients after accepting the screened Chinese herbal extracts. FIG. 4C shows the results of the immunity value of ovarian cancer patients after accepting the screened Chinese herbal extracts. FIG. 4D shows the results of the immunity value of colorectal cancer patients after accepting the screened Chinese herbal extracts. In FIG. 4A to FIG. 4D, each of the dashed lines represents the immunity trend of a single subject before and after accepting the screened Chinese herbal extract, wherein “pre-test” means the immunity value of the subject before accepting the screened Chinese herbal extract, and “post-test” means the immunity value of the subject after accepting the screened Chinese herbal extract. Furthermore, an integral immunity trend of all the subjects is shown by the thick line, and the medians of the immunity values before and after accepting the screened Chinese herbal extracts thereof are marked by circles. Unless otherwise specified, if the definitions of the marks in the following figures are the same as those shown in FIG. 4A to FIG. 4D, it will not be described again.

As shown in FIG. 4A, the median of immunity value of the breast cancer patients before accepting the screened Chinese herbal extracts is 2750 μg/mL, and the median of immunity value thereof after accepting the screened Chinese herbal extracts significantly increases to 3300 μg/mL (p<0.001). It is shown that the Chinese herbal extract screened from the Chinese herb combination by the using method of immunity detecting kit of the present disclosure has the immunity enhancing activity for the breast cancer patients and can effectively increase the immunity of the breast cancer patients.

As shown in FIG. 4B, the median of immunity value of the endometrial cancer patients before accepting the screened Chinese herbal extracts is 2400 μg/mL, and the median of immunity value thereof after accepting the screened Chinese herbal extracts significantly increases to 2950 μg/mL (p<0.001). It is shown that the Chinese herbal extract screened from the Chinese herb combination by the using method of immunity detecting kit of the present disclosure has the immunity enhancing activity for the endometrial cancer patients and can effectively increase the immunity of the endometrial cancer patients.

As shown in FIG. 4C, the median of immunity value of the ovarian cancer patients before accepting the screened Chinese herbal extracts is 2300 μg/mL, and the median of immunity value thereof after accepting the screened Chinese herbal extracts significantly increases to 2700 μg/mL (p=0.13). It is shown that the Chinese herbal extract screened from the Chinese herb combination by the using method of immunity detecting kit of the present disclosure has the immunity enhancing activity for the ovarian cancer patients and can effectively increase the immunity of the ovarian cancer patients.

As shown in FIG. 4D, the median of immunity value of the colorectal cancer patients before accepting the screened Chinese herbal extracts is 2475 μg/mL, and the median of immunity value thereof after accepting the screened Chinese herbal extracts significantly increases to 3350 μg/mL (p=0.007). It is shown that the Chinese herbal extract screened from the Chinese herb combination by the using method of immunity detecting kit of the present disclosure has the immunity enhancing activity for the colorectal cancer patients and can effectively increase the immunity of the colorectal cancer patients.

2. Analysis of the Body Constitutions in Traditional Chinese Medicine of the Patients Suffering from Cancer

In the present experiment, the body constitutions in traditional Chinese medicine of the 218 patients suffering from cancer are analyzed so as to compare the types of the body constitutions in traditional Chinese medicine of the patients suffering from cancer and then assess the relationship between the immunity of the patients suffering from cancer and the body constitutions in traditional Chinese medicine thereof. In detail, the body constitutions in traditional Chinese medicine can be defined to nine types, namely Gentleness constitution, Qi-deficiency constitution, Phlegm-dampness constitution, Qi-depression constitution, Blood-stasis constitution, Yin-deficiency constitution,

Yang-deficiency constitution, Dampness-heat constitution and Special diathesis constitution base on the physical condition of the individual. Gentleness constitution is characterized by having a rosy complexion, good mental state, and good sleep. Qi-deficiency constitution is characterized by easy sweating, lack of energy, and loss of muscles. Phlegm-dampness constitution is characterized by chest tightness, easy phlegm in the throat, and obesity. Qi-depression constitution is characterized by depression, having foreign body sensation in the throat, and swelling and pain on both sides of the chest. Blood-stasis constitution is characterized by dark skin, dark lip and petechiae on the tongue. Yin-deficiency constitution is characterized by being thirsty in the mouth, nose and throat, feeling warm in the palms and soles of the feet, and having insomnia. Yang-deficiency constitution is characterized by pale face, body chills, and edema. Dampness-heat constitution is characterized by being thirsty and fatigue. Special diathesis constitution is characterized by being easily allergic to external stimulation.

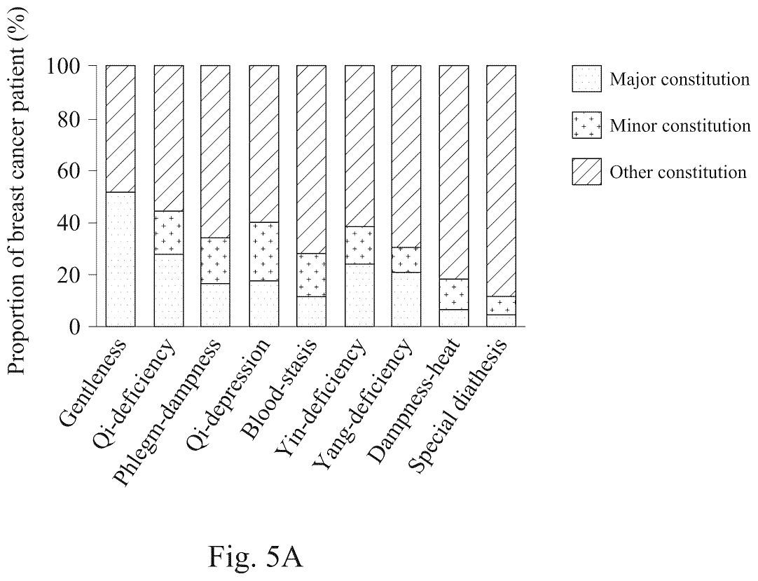

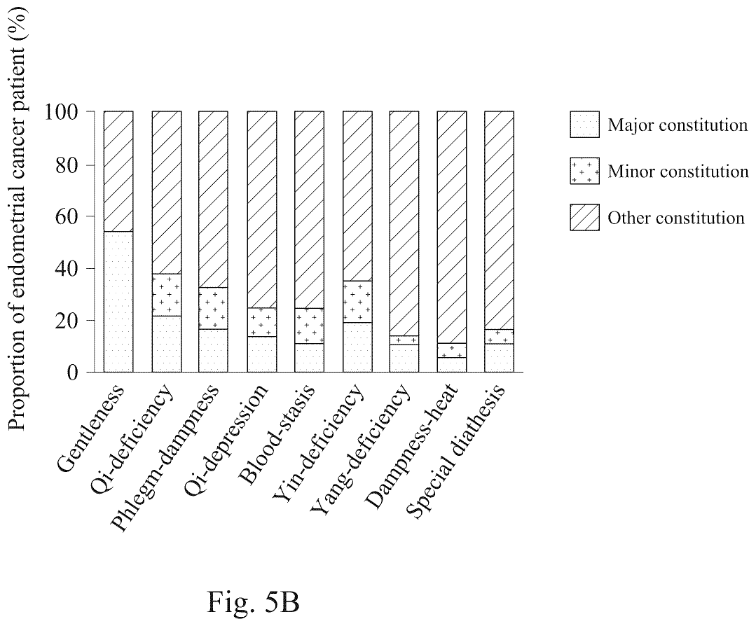

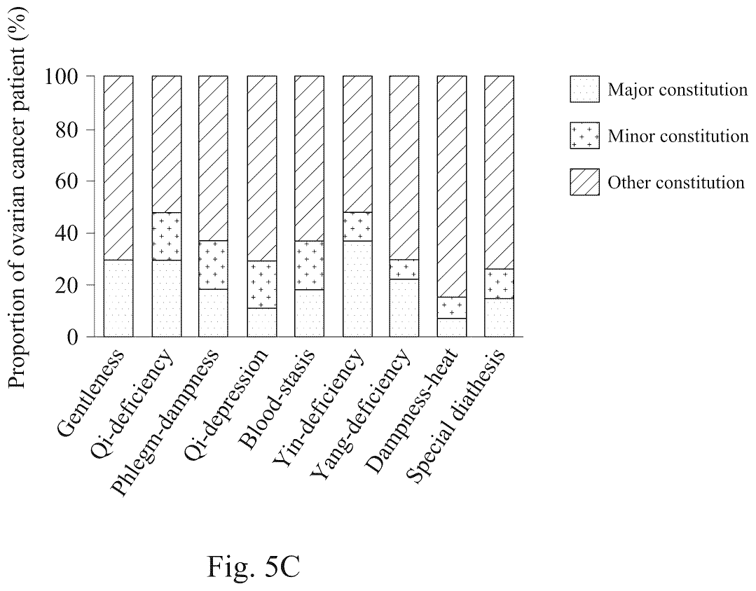

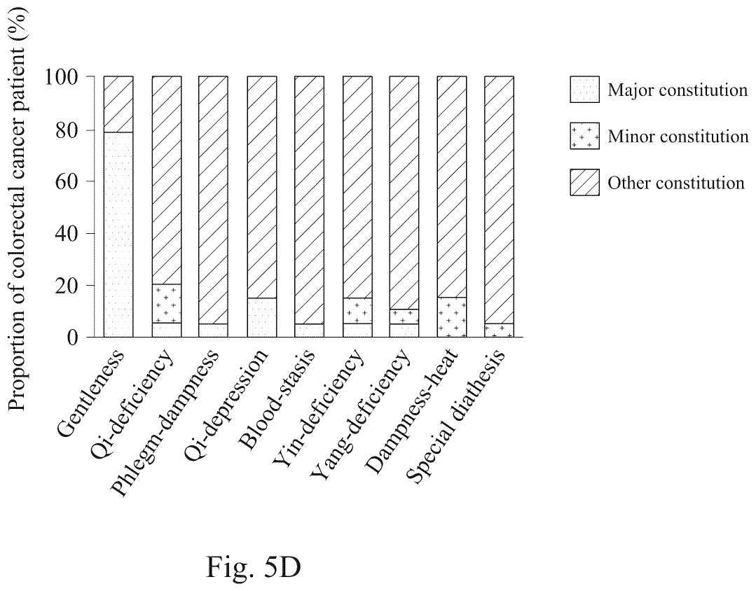

Reference is made to FIG. 5A, which shows the proportion distribution of the breast cancer patients with different body constitutions in traditional Chinese medicine, and the proportions of the nine body constitutions in traditional Chinese medicine of the breast cancer patients are shown in Table 1.

TABLE 1

| | Major | Minor | Other |
|---|---|---|---|
| | constitution | constitution | constitution |
| | (%) | (%) | (%) |
| Gentleness | 52.17 | — | 47.83 |
| constitution | | | |
| Qi-deficiency | 28.26 | 15.94 | 55.80 |
| constitution | | | |
| Phlegm-dampness | 16.67 | 17.39 | 65.94 |
| constitution | | | |
| Qi-depression | 18.12 | 21.74 | 60.14 |
| constitution | | | |
| Blood-stasis | 11.59 | 16.67 | 71.74 |
| constitution | | | |
| Yin-deficiency | 23.91 | 14.49 | 61.59 |
| constitution | | | |
| Yang-deficiency | 21.01 | 9.42 | 69.57 |
| constitution | | | |
| Dampness-heat | 6.52 | 11.59 | 81.88 |
| constitution | | | |
| Special diathesis | 4.35 | 7.25 | 88.41 |
| constitution | | | |

In detail, in the nine body constitutions in traditional Chinese medicine, except for Gentleness constitution, the other eight body constitutions in traditional Chinese medicine can be existed at the same time. In other words, the subject can have more than one body constitution in traditional Chinese medicine, and the symptoms of the major one of the body constitutions in traditional Chinese medicine are shown.

As shown in FIG. 5A and Table 1, within 138 breast cancer patients, the breast cancer patients having Gentleness constitution are the most, and the proportion thereof is 52.17%. The second is the breast cancer patients having Qi-deficiency constitution with the proportion thereof being 28.26%, and the third is the breast cancer patients having Yin-deficiency constitution with the proportion thereof being 23.91%.

Reference is made to FIG. 5B, which shows the proportion distribution of the endometrial cancer patients with different body constitutions in traditional Chinese medicine, and the proportions of the nine body constitutions in traditional Chinese medicine of the endometrial cancer patients are shown in Table 2.

TABLE 2

| | Major | Minor | Other |
|---|---|---|---|
| | constitution | constitution | constitution |
| | (%) | (%) | (%) |
| Gentleness | 57.14 | — | 42.86 |
| constitution | | | |
| Qi-deficiency | 22.86 | 17.14 | 60 |
| constitution | | | |
| Phlegm-dampness | 17.14 | 17.14 | 65.72 |
| constitution | | | |
| Qi-depression | 14.29 | 11.43 | 74.28 |
| constitution | | | |
| Blood-stasis | 11.43 | 14.29 | 74.28 |
| constitution | | | |
| Yin-deficiency | 20 | 17.14 | 62.86 |
| constitution | | | |
| Yang-deficiency | 11.43 | 2.86 | 85.71 |
| constitution | | | |
| Dampness-heat | 5.71 | 5.71 | 88.57 |
| constitution | | | |
| Special diathesis | 11.43 | 5.71 | 82.86 |
| constitution | | | |

As shown in FIG. 5B, within 35 endometrial cancer patients, the endometrial cancer patients having Gentleness constitution are the most, and the proportion thereof is 57.14%. The second is the endometrial cancer patients having Qi-deficiency constitution with the proportion thereof being 22.86%, the third is the endometrial cancer patients having Yin-deficiency constitution with the proportion thereof being 20%, and the fourth is the endometrial cancer patients having Phlegm-dampness constitution with the proportion thereof being 17.14%.

Reference is made to FIG. 5C, which shows the proportion distribution of the ovarian cancer patients with different body constitutions in traditional Chinese medicine, and the proportions of the nine body constitutions in traditional Chinese medicine of the ovarian cancer patients are shown in Table 3.

TABLE 3

| | Major | Minor | Other |
|---|---|---|---|
| | constitution | constitution | constitution |
| | (%) | (%) | (%) |
| Gentleness | 31 | — | 69 |
| constitution | | | |
| Qi-deficiency | 31 | 19 | 50 |
| constitution | | | |
| Phlegm-dampness | 19 | 19 | 62 |
| constitution | | | |
| Qi-depression | 12 | 19 | 69 |
| constitution | | | |
| Blood-stasis | 19 | 19 | 62 |
| constitution | | | |
| Yin-deficiency | 38 | 12 | 50 |
| constitution | | | |
| Yang-deficiency | 23 | 8 | 69 |
| constitution | | | |
| Dampness-heat | 8 | 8 | 85 |
| constitution | | | |
| Special diathesis | 15 | 12 | 73 |
| constitution | | | |

As shown in FIG. 5C, within 26 ovarian cancer patients, the ovarian cancer patients having Yin-deficiency constitution are the most, and the proportion thereof is 38%. The second is the ovarian cancer patients having Gentleness constitution with the proportion thereof being 31%, and the third is the ovarian cancer patients having Qi-deficiency constitution with the proportion thereof being 31%.

Reference is made to 5D, which shows the proportion distribution of the colorectal cancer patients with different body constitutions in traditional Chinese medicine, and the proportions of the nine body constitutions in traditional Chinese medicine of the colorectal cancer patients are shown in Table 4.

TABLE 4

| | Major | Minor | Other |
|---|---|---|---|
| | constitution | constitution | constitution |
| | (%) | (%) | (%) |
| Gentleness | 78.95 | — | 21.05 |
| constitution | | | |
| Qi-deficiency | 5.26 | 15.79 | 78.95 |
| constitution | | | |
| Phlegm-dampness | 5.26 | — | 94.74 |
| constitution | | | |
| Qi-depression | 15.79 | — | 84.21 |
| constitution | | | |
| Blood-stasis | 5.26 | — | 94.74 |
| constitution | | | |
| Yin-deficiency | 5.26 | 10.53 | 84.21 |
| constitution | | | |
| Yang-deficiency | 5.26 | 5.26 | 89.48 |
| constitution | | | |
| Dampness-heat | — | 15.79 | 84.21 |
| constitution | | | |
| Special diathesis | — | 5.26 | 94.74 |
| constitution | | | |

As shown in FIG. 5D, within 19 colorectal cancer patients, the colorectal cancer patients having Gentleness constitution are the most, and the proportion thereof is 78.95%.

As shown above, the subjects suffering from the same disease can have different body constitutions in traditional Chinese medicine and have different compositions thereof. Hence, by assessing the immune activity of the screened Chinese herbal extracts of the present disclosure for the immune cells and analyzing the body constitutions in traditional Chinese medicine of the individual as an auxiliary, it is favorable for screening the Chinese herbal extracts that are suitable for the current physical condition of the individual, and thus it is favorable for facilitating the formulation of the personal treatment strategy.

### 3. Analysis of the Relationship Between the Immunity of the Breast Cancer Patients and the Body Constitutions in Traditional Chinese Medicine Thereof

In the present experiment, the first results and the second results of the blood samples of the 138 breast cancer patients are analyzed so as to screen the Chinese herbal extracts of the Chinese herb combination having the immunity enhancing activity based on the physical condition of each of the breast cancer patients. The screened Chinese herbal extracts suitable for different breast cancer patients are respectively applied to the breast cancer patients for two weeks, and the expressed amounts of TNF-α, IFN-γ and IL-2 after accepting the screened Chinese herbal extracts are detected so as to assess the degrees of immunity of the breast cancer patients having different body constitutions in traditional Chinese medicine.

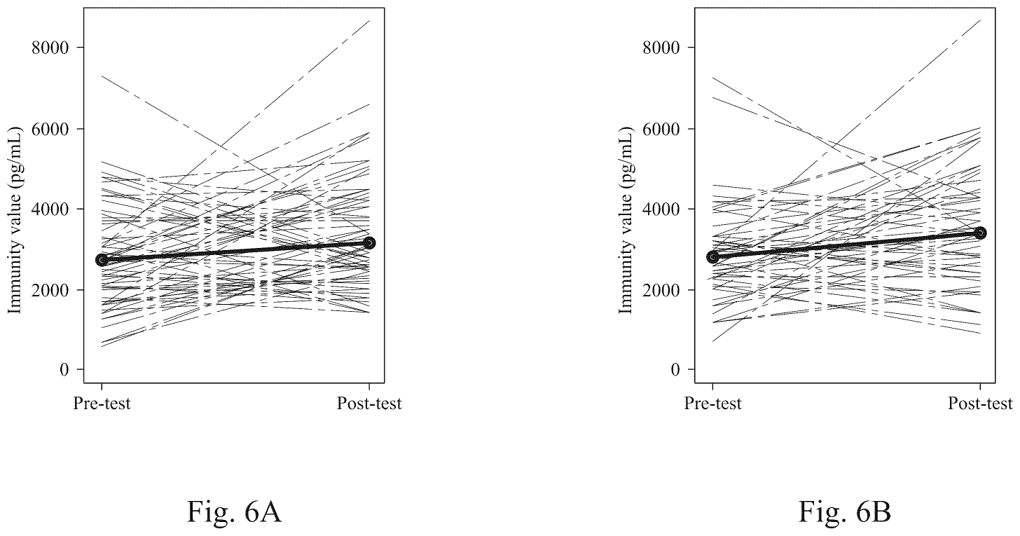

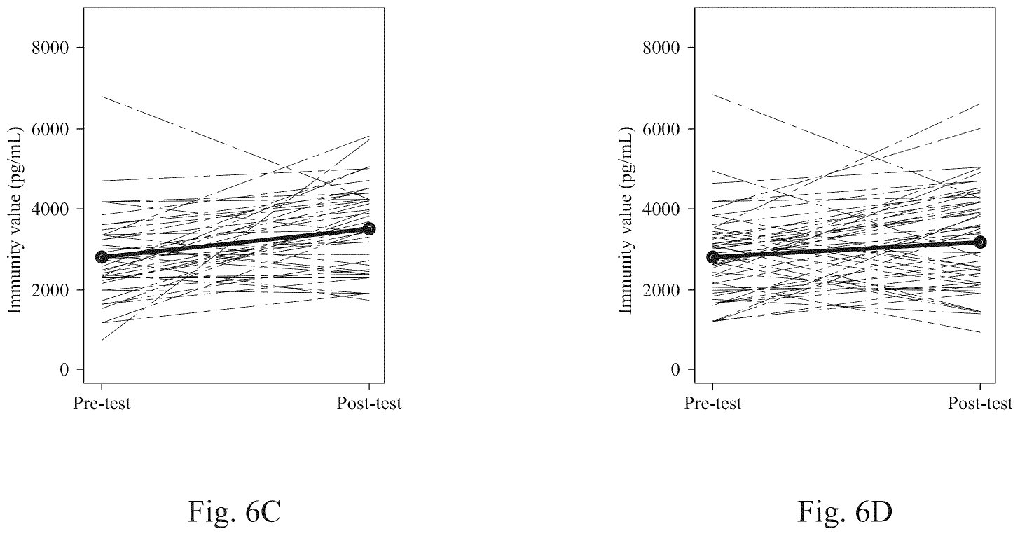

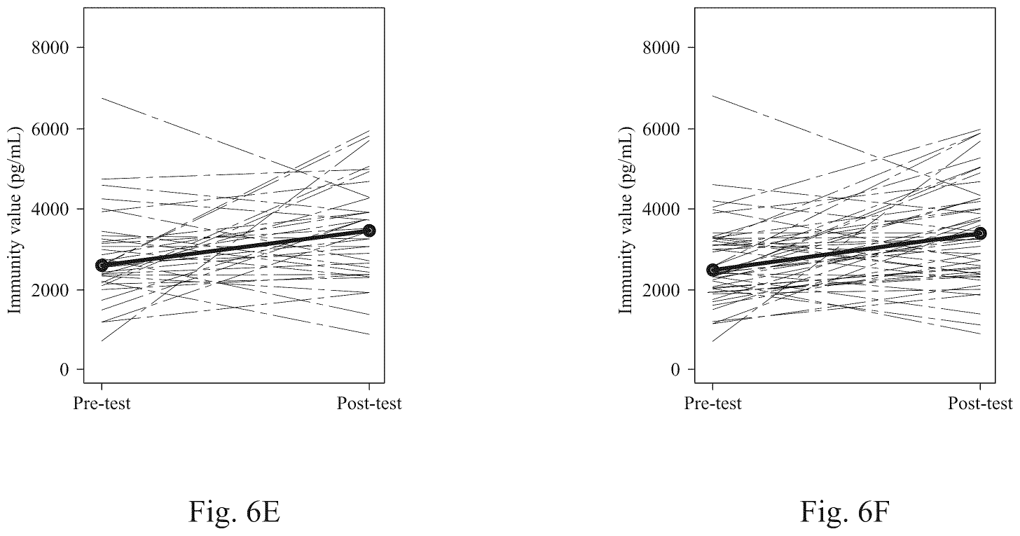

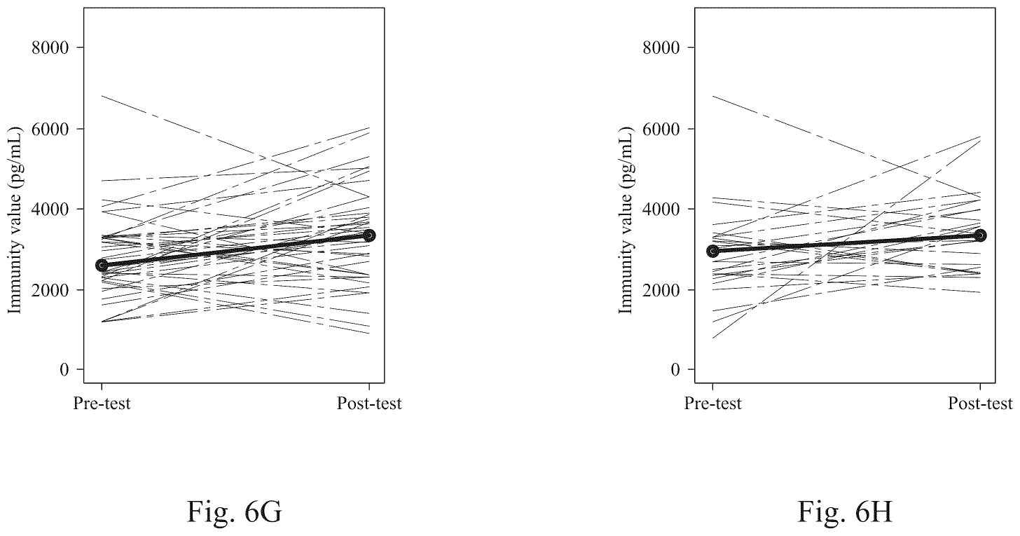

Reference is made to FIG. 6A, which shows the results of the immunity value of the breast cancer patients with Gentleness constitution after accepting the screened Chinese herbal extracts. In detail, as shown in FIG. 6A, the median of immunity value of the breast cancer patients having Gentleness constitution before accepting the screened Chinese herbal extracts is 2750 μg/mL, and the median of immunity value after accepting the screened Chinese herbal extracts significantly increases to 3150 μg/mL (p=0.014).

Reference is made to FIG. 6B, which shows the results of the immunity value of the breast cancer patients with Qi-deficiency constitution after accepting the screened Chinese herbal extracts. In detail, as shown in FIG. 6B, the median of immunity value of the breast cancer patients having Qi-deficiency constitution before accepting the screened Chinese herbal extracts is 2800 μg/mL, and the median of immunity value after accepting the screened Chinese herbal extracts significantly increases to 3400 μg/mL (p=0.002).

Reference is made to FIG. 6C, which shows the results of the immunity value of the breast cancer patients with Phlegm-dampness constitution after accepting the screened Chinese herbal extracts. In detail, as shown in FIG. 6C, the median of immunity value of the breast cancer patients having Phlegm-dampness constitution before accepting the screened Chinese herbal extracts is 2800 μg/mL, and the median of immunity value after accepting the screened Chinese herbal extracts significantly increases to 3500 μg/mL (p=0.001).

Reference is made to FIG. 6D, which shows the results of the immunity value of the breast cancer patients with Qi-depression constitution after accepting the screened Chinese herbal extracts. In detail, as shown in FIG. 6D, the median of immunity value of the breast cancer patients having Qi-depression constitution before accepting the screened Chinese herbal extracts is 2800 μg/mL, and the median of immunity value after accepting the screened Chinese herbal extracts significantly increases to 3200 μg/mL (p=0.018).

Reference is made to FIG. 6E, which shows the results of the immunity value of the breast cancer patients with Blood-stasis constitution after accepting the screened Chinese herbal extracts. In detail, as shown in FIG. 6E, the median of immunity value of the breast cancer patients having Blood-stasis constitution before accepting the screened Chinese herbal extracts is 2600 μg/mL, and the median of immunity value after accepting the screened Chinese herbal extracts significantly increases to 3450 μg/mL (p=0.006).

Reference is made to FIG. 6F, which shows the results of the immunity value of the breast cancer patients with Yin-deficiency constitution after accepting the screened Chinese herbal extracts. In detail, as shown in FIG. 6F, the median of immunity value of the breast cancer patients having Yin-deficiency constitution before accepting the screened Chinese herbal extracts is 2500 μg/mL, and the median of immunity value after accepting the screened Chinese herbal extracts significantly increases to 3400 μg/mL (p=0.001).

Reference is made to FIG. 6G, which shows the results of the immunity value of the breast cancer patients with Yang-deficiency constitution after accepting the screened Chinese herbal extracts. In detail, as shown in FIG. 6G, the median of immunity value of the breast cancer patients having Yang-deficiency constitution before accepting the screened Chinese herbal extracts is 2600 μg/mL, and the median of immunity value after accepting the screened Chinese herbal extracts significantly increases to 3350 μg/mL (p=0.004).

Reference is made to FIG. 6H, which shows the results of the immunity value of the breast cancer patients with Dampness-heat constitution after accepting the screened Chinese herbal extracts. In detail, as shown in FIG. 6H, the median of immunity value of the breast cancer patients having Dampness-heat constitution before accepting the screened Chinese herbal extracts is 2950 μg/mL, and the median of immunity value after accepting the screened Chinese herbal extracts significantly increases to 3400 μg/mL (p=0.057).

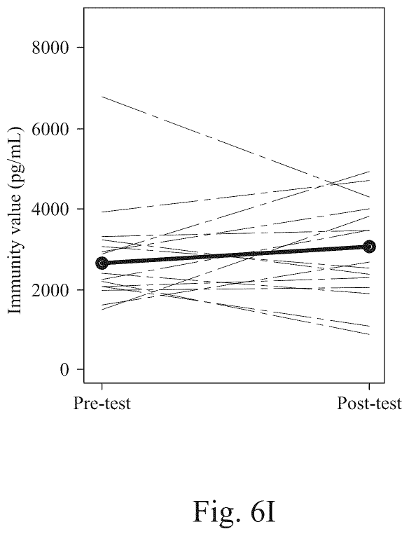

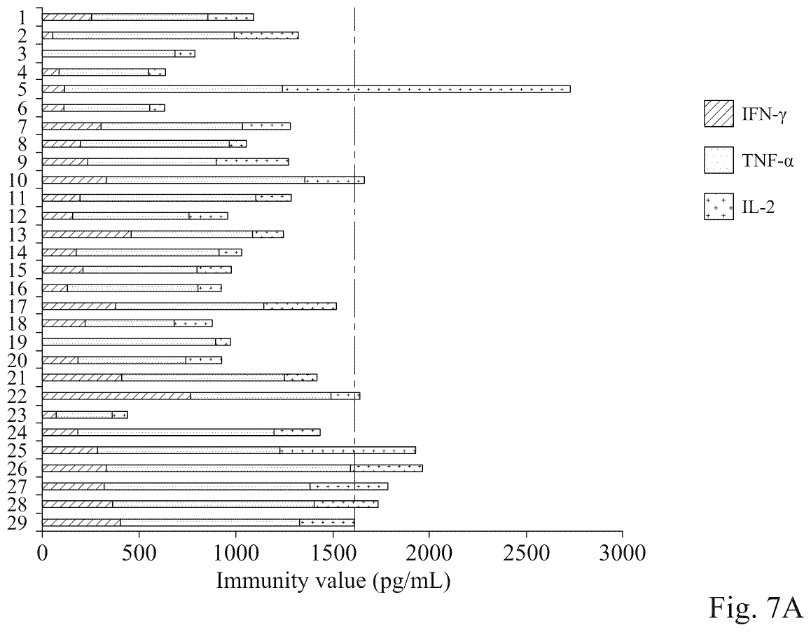

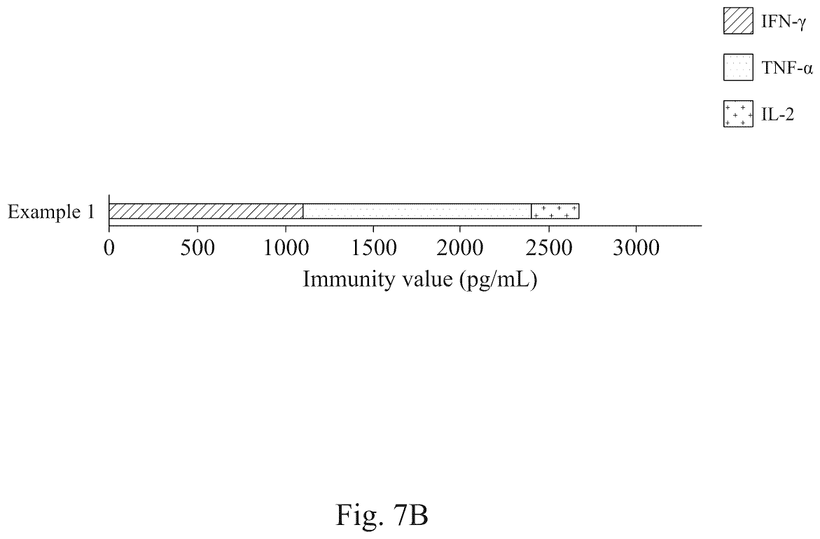

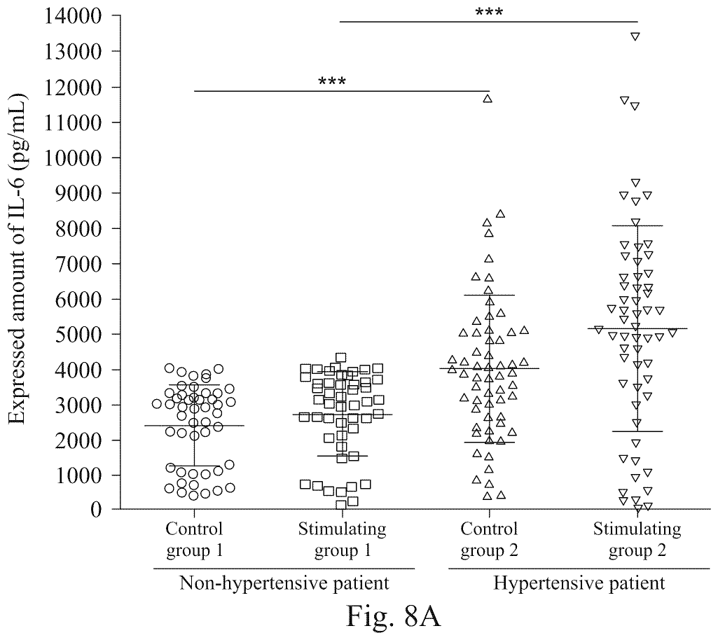

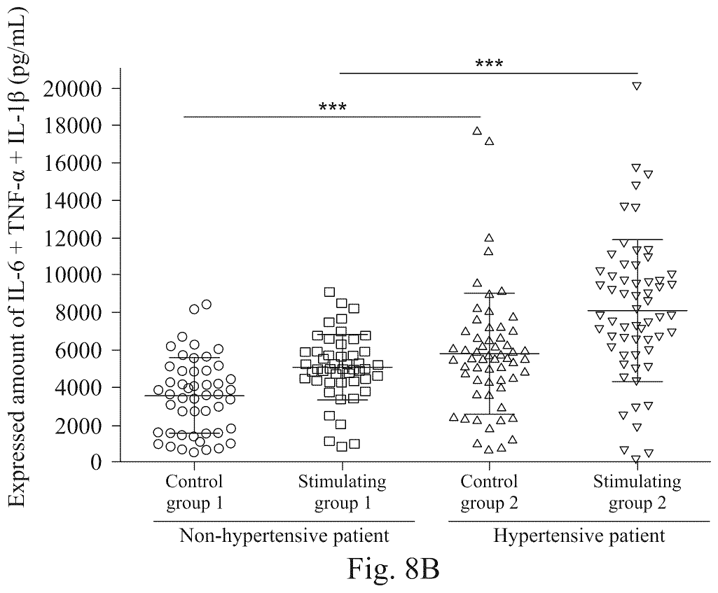

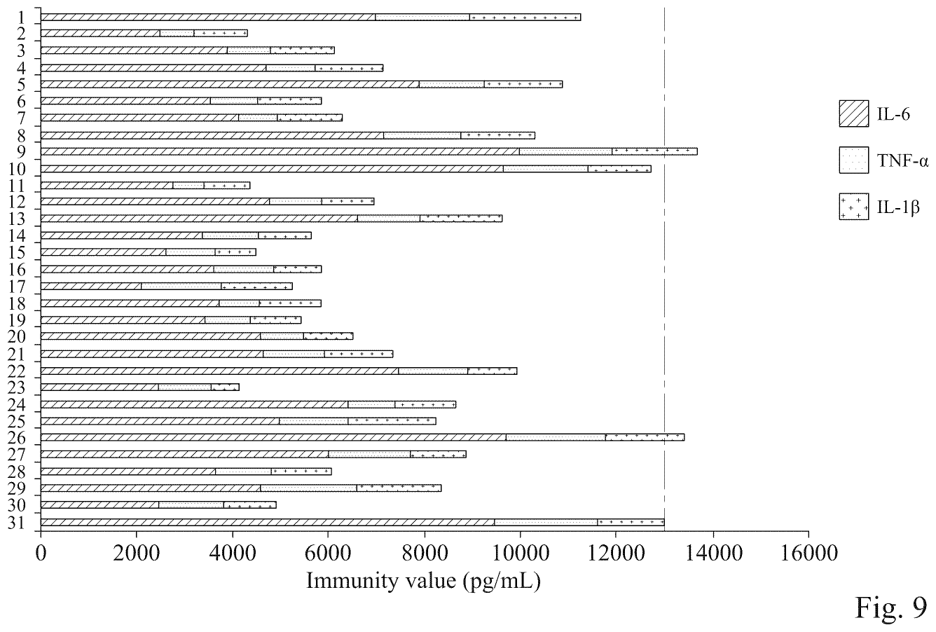

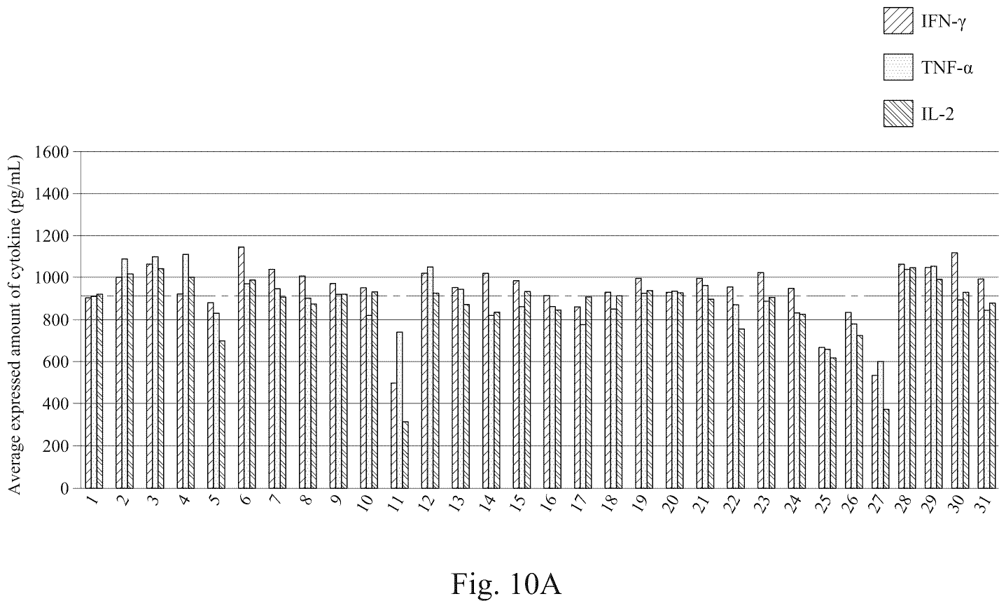

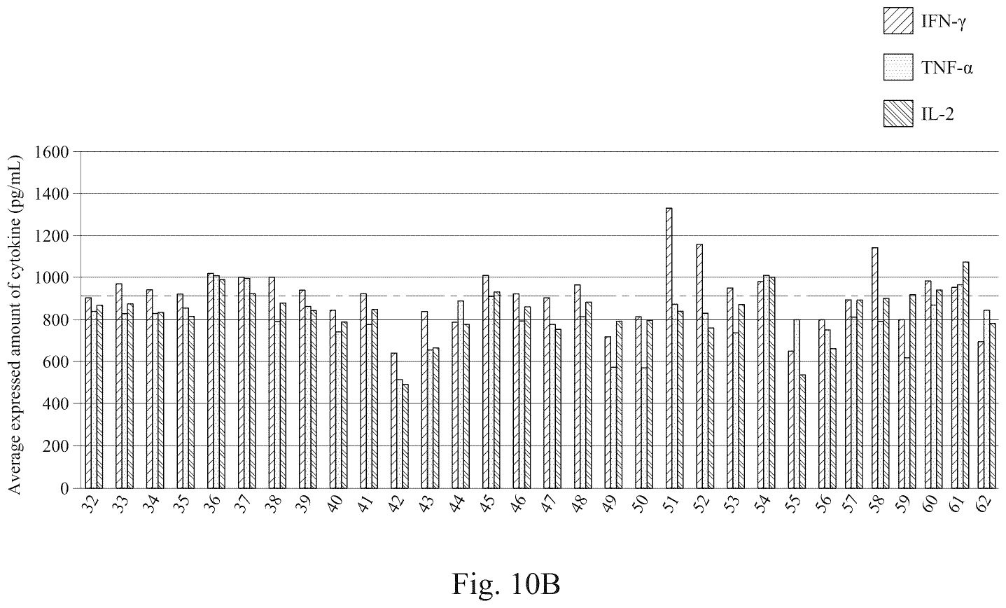

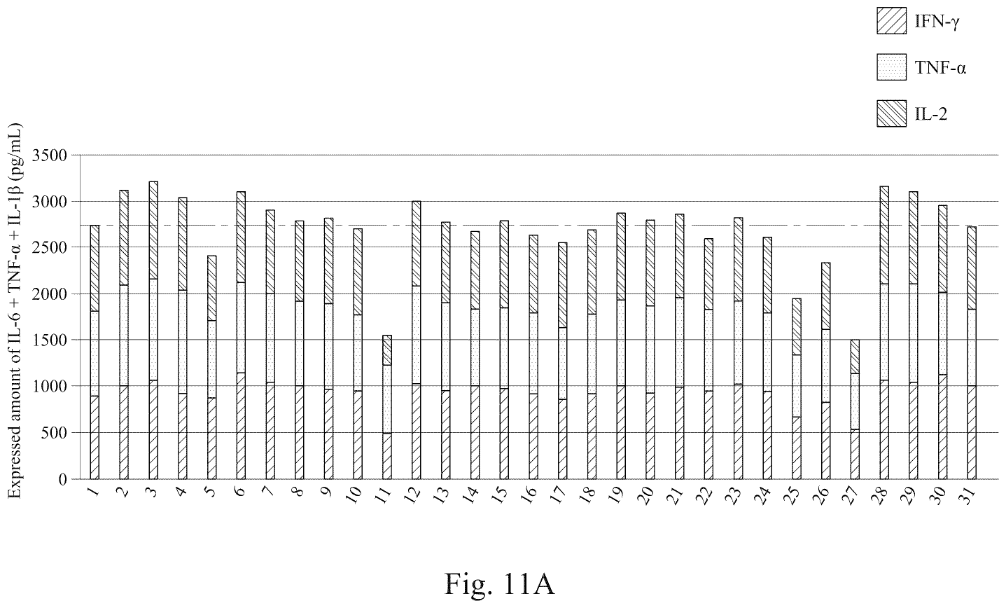

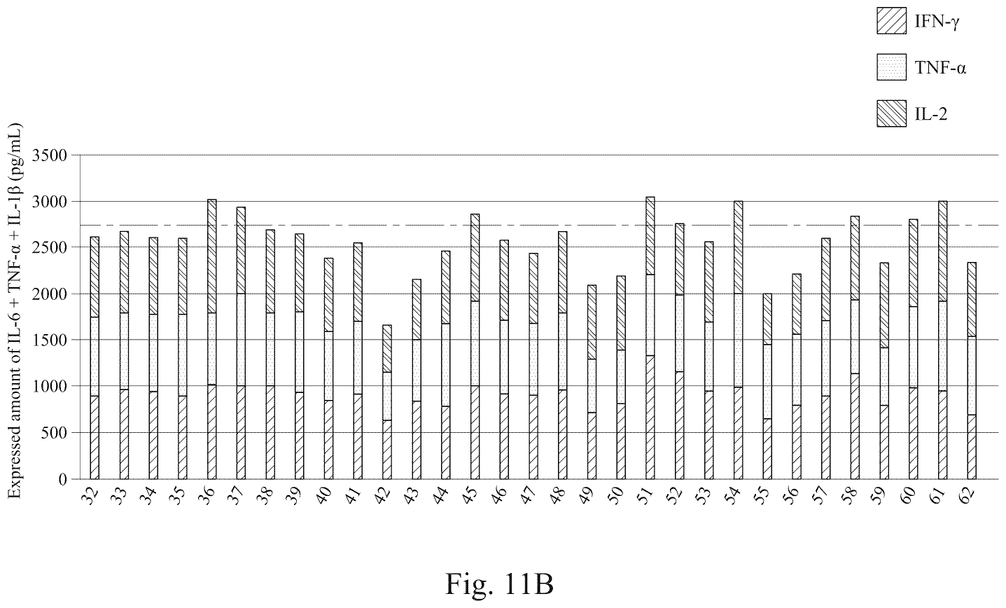

Reference is made to FIG. 61, which shows the results of the immunity value of the breast cancer patients with Special diathesis constitution after accepting the screened Chinese herbal extracts. In detail, as shown in FIG. 61, the median of immunity value of the breast cancer patients having Special diathesis constitution before accepting the screened Chinese herbal extracts is 2650 μg/mL, and the median of immunity value after accepting the screened Chinese herbal extracts significantly increases to 3062 μg/mL (p=0.5).

As shown above, the screened Chinese herbal extracts screened from the Chinese herb combination by the using method of immunity detecting kit of the present disclosure can have the immunity enhancing activity for the breast cancer patients having different body constitutions, and thus the immunity of the breast cancer patients can be effectively improved. Therefore, the immunity detecting kit and the using method of immunity detecting kit of the present disclosure can be used to detect the immune status of the subjects having different body constitutions and suffering from different diseases so as to effectively increase the immunity of the immunocompromised subjects by the screened Chinese herbal extracts.

### 4. Analysis of the Effects of Different Chinese Herbal Extracts to the Immunity of the Breast Cancer Patients

In the present experiment, the blood sample of one of the 138 breast cancer patients is selected as Example 1, and the first result and the second result thereof are compared so as to assess the effects of the screened Chinese herbal extracts on the immunity of the breast cancer patients.

Reference is made to FIG. 7A, which shows the results of the immunity value of the breast cancer patient of Example 1 in response to different Chinese herbal extracts. The numbers shown in the vertical axis of FIG. 7A represents the Chinese herbal extracts, and the numbers corresponding to the Chinese herbal extracts are listed in Table 5, wherein the dashed line is the baseline according to the first result (Number 29), and “Basement” shown in Table 5 represents the group by treating the immune cells with the phorbol 12-myristate 13-acetate and the endotoxin inhibitor.

TABLE 5

| No. | Name | No. | Name |
|---|---|---|---|
| 1 | Scutellariae Radix | 2 | Massa Medicata Fermentata |
| 3 | Spreading Hedyotis Herb | 4 | Scutellariae Barbatae Herba |
| 5 | Schisandrae Chinensis Fructus | 6 | Saposhnikoviae Radix |
| 7 | Dioscoreae Rhizoma | 8 | Reynoutriae Multiflorae |
| | | | Radix |
| 9 | Polygonati Rhizoma | 10 | Polyporus |
| 11 | Achyranthis Bidentatae Radix | 12 | Lycii Fructus |
| 13 | Jujubae Fructus | 14 | Salviae Miltiorrhizae |
| | | | Radix et Rhizoma |
| 15 | Panacis Quinquefolii Radix | 16 | Ginseng Radix et Rhizoma |
| 17 | Curcumae Longae Rhizoma | 18 | Zingiberis Rhizoma |
| 19 | Cinnamomi Cortex | 20 | Astragali Radix |
| 21 | Rehmanniae Radix Praeparata | 22 | Paeoniae Radix Alba |
| 23 | Chuanxiong Rhizoma | 24 | Angelicae Sinensis Radix |
| 25 | Glycyrrhizae Radix et | 26 | Poria |
| | Rhizoma | | |
| 27 | Atractylodis Macrocephalae | 28 | Codonopsis Radix |
| | Rhizoma | | |
| 29 | Basement | | |

As shown in FIG. 7A and Table 5, the basic immunity value of the breast cancer patient of Example 1 (Number 29) is 1615.91 pg/mL, and the Schisandrae Chinensis Fructus extract (Number 5), the Glycyrrhizae Radix et Rhizoma extract (Number 25), the Poria extract (Number 26), the Atractylodis Macrocephalae Rhizoma extract (Number 27) and the Codonopsis Radix extract (Number 28) have the immunity enhancing activity for the breast cancer patient of Example 1, wherein the immunity enhancing activity of the Schisandrae Chinensis Fructus extract is higher than those of other Chinese herbal extracts.

Reference is made to FIG. 7B, which shows the results of the immunity value of the breast cancer patient of Example 1 after accepting the Schisandrae Chinensis Fructus extract for two weeks. As shown in FIG. 7B, after accepting the Schisandrae Chinensis Fructus extract for two weeks, the basic immunity value of the breast cancer patient of Example 1 increases from 1615.91 pg/mL to 2674.26 pg/mL. Therefore, it is shown that the screened Chinese herbal extracts screened from the Chinese herb combination by the using method of immunity detecting kit of the present disclosure have the immunity enhancing activity for the immunocompromised subject and can be used to improve the immunity thereof.

### II. Analysis of the Immunity of the Hypertensive Patients

### 1. Analysis of the Relationship Between the Gender and the Immunity of the Hypertensive Patients

In the present experiment, the blood samples of the hypertensive patients are analyzed by the using method 100 of immunity detecting kit of the present disclosure so as to compare the first results of the blood samples of male hypertensive patients with the first results of the blood samples of female hypertensive patients and then analyze the relationship between the gender and immunity of the hypertensive patients. In the present experiment, the immune-stimulant agent of the immunity detecting kit is the lipopolysaccharide, wherein the concentration of the lipopolysaccharide is 200 ng/ml, the first culturing time is 24 hours, and the cytokine in the first culturing mixture includes TNF-α, IL-6 and IL-1B.

In detail, the blood samples of 605 hypertensive patients including 348 male hypertensive patients and 257 female hypertensive patients are analyzed in the present experiment, and the immunity values of the first results of the blood samples of the male hypertensive patients and the first results of the blood samples of the female hypertensive patients are shown in Table 6.

TABLE 6

| | IL-6 + TNF-α + | | | |
|---|---|---|---|---|
| | IL-1β | IL-6 | TNF-α | IL-1β |
| | (pg/mL) | (pg/mL) | (pg/mL) | (pg/mL) |
| Male + | 8579 ± 3569 | 5959 ± 3079 | 1090 ± 924 | 1529 ± 842 |
| Female | | | | |
| Male | 8871 ± 3568 | 6076 ± 2965 | 1172 ± 961 | 1623 ± 902 |
| Female | 8131 ± 3531 | 5780 ± 3245 | 966 ± 850 | 1386 ± 717 |
| p value | 0.018 | 0.274 | 0.009 | 0.001 |

As shown in Table 6, within all the hypertensive patients, the average expressed amount of IL-6 is 5959 μg/mL, the average expressed amount of TNF-α is 1090 μg/mL, and the average expressed amount of IL-1B is 1529 μg/mL. Further, compared with the sum of the average expressed amounts of IL-6, TNF-α and IL-1B of the male hypertensive patients, the sum of the average expressed amounts of IL-6, TNF-α and IL-1B of the female hypertensive patients is smaller. It is shown that the inflammatory response of the female hypertensive patients is milder than that of the male hypertensive patients.

### 2. Analysis of the Expressed Amounts of Inflammation-Related Cytokines of the Non-Hypertensive Patients and the Hypertensive Patients

In the present experiment, the blood samples of 49 non-hypertensive patients and 60 hypertensive patients are analyzed by the using method 100 of immunity detecting kit of the present disclosure so as to compare the first results of the blood samples of the non-hypertensive patients with the first results of the blood samples of the hypertensive patients and then assess the degree of inflammatory response of the hypertensive patients. In the present experiment, the immune-stimulant agent of the immunity detecting kit is lipopolysaccharide, the concentration of lipopolysaccharide is 200 ng/ml, the first culturing time is 24 hours, and the cytokine of the first culturing mixture includes TNF-α, IL-6 and IL-1B. Control group 1, Stimulating group 1, Control group 2 and Stimulating group 2 are used in the present experiment, wherein Control group 1 includes the cytokine secreted by the immune cells without the stimulation of the lipopolysaccharide in the blood sample of the non-hypertensive patients, Stimulating group 1 includes the cytokine secreted by the immune cells with the stimulation of the lipopolysaccharide in the blood sample of the non-hypertensive patients, Control group 2 includes the cytokine secreted by the immune cells without the stimulation of the lipopolysaccharide in the blood sample of the hypertensive patients, and Stimulating group 2 includes the cytokine secreted by the immune cells with the stimulation of the lipopolysaccharide in the blood sample of the hypertensive patients.

Reference is made to FIG. 8A and FIG. 8B. FIG. 8A shows the expressed amounts of IL-6 of the non-hypertensive patients and the hypertensive patients. FIG. 8B shows the sum of the expressed amounts of IL-6, TNF-α and IL-1B of the non-hypertensive patients and the hypertensive patients. In detail, in FIG. 8A and FIG. 8B, the mark “***” represents that the data is statistically significant compared with Control group 1 or Stimulating group 1 (p<0.001).

As shown in FIG. 8A, compared with the expressed amount of IL-6 of Control group 1, Control group 2 has a significantly higher expressed amount of IL-6, and compared with the expressed amount of IL-6 of Stimulating group 1, Stimulating group 2 has a significantly higher expressed amount of IL-6. As shown in FIG. 8B, compared with the sum of the expressed amounts of IL-6, TNF-α and IL-1B of Control group 1, the sum of the expressed amounts of IL-6, TNF-α and IL-1B of Control group 2 is significantly higher, and compared with the sum of the expressed amounts of IL-6, TNF-α and IL-1B of Stimulating group 1, the sum of the expressed amounts of IL-6, TNF-α and IL-1B of Stimulating group 2 is significantly higher.

As shown above, the immunity of the hypertensive patients is stronger than that of the non-hypertensive patients, resulting in the chronic inflammation of the hypertensive patients. Therefore, by screening and accepting the Chinese herbal extracts having the immunity suppressing activity, the physical condition with the overactive immune system of the hypertensive patients can be improved.

### 3. Analysis of the Effects of the Chinese Herbal Extracts on the Immunity of the Hypertensive Patients

In the present experiment, the blood sample of one of the 60 hypertensive patients is selected as Example 2, and the first result and the second result thereof are compared so as to assess the effects of the screened Chinese herbal extracts on the immunity of the hypertensive patients.

Reference is made to FIG. 9, which shows the results of the immunity value of the hypertensive patient of Example 2 in response to different Chinese herbal extracts. In detail, “Immunity value” shown in the horizontal axis of FIG. 9 represents the sum of the expressed amounts of IL-6, TNF-α and IL-1B, and the numbers shown in the vertical axis thereof represent the Chinese herbal extracts shown in Table 7. Further, the dashed line shown in FIG. 9 is the baseline according to the first result (Number 31), and “Basement” shown in Table 7 represents the group by treating the immune cells with the lipopolysaccharide.

TABLE 7

| No. | Name | No. | Name |
|---|---|---|---|
| 1 | Saposhnikoviae Radix | 2 | Salviae Miltiorrhizae |
| | | | Radix et Rhizoma |
| 3 | Cyathulae Radix | 4 | Anemarrhenae Rhizoma |
| 5 | Moutan Radicis Cortex | 6 | Lycii Radicis Cortex |
| 7 | Siegesbeckiae Herba | 8 | Taxilli Herba |
| 9 | Gentianae Macrophyllae | 10 | Poria |
| | Radix | | |
| 11 | Artemisiae Scopariae | 12 | Reynoutriae Rhizoma |
| | Herba | | et Radix |
| 13 | Plantaginis Semen | 14 | Taraxaci Herba |
| 15 | Lonicerae Japonicae Flos | 16 | Houttuyniae Herba |
| 17 | Isatidis Radix | 18 | Rhei Radix et Rhizoma |
| 19 | Cassiae Semen | 20 | Phellodendri Cortex (including |
| | | | the extract of Phellodron |
| | | | chinense Schneid.) |
| 21 | Isatidis Folium | 22 | Gentianae Radix et Rhizoma |
| 23 | Scutellariae Radix | 24 | Forsythiae Fructus |
| 25 | Coptidis Rhizoma | 26 | Gardeniae Fructus |
| 27 | Long-Dan-Xie-Gan-Tang | 28 | Yin-Chen-Hao-Tang |
| 29 | Ban-Xia-Xie-Xin-Tang | 30 | San-Huang-Xie-Xin-Tang |
| 31 | Basement | | |

As shown in FIG. 9 and Table 7, the basic immunity value of the hypertensive patient of Example 2 (Number 31) is 12991.22 pg/mL, and the Scutellariae Radix extract (Number 23) has an excellent immunity suppressing activity for the hypertensive patient of Example 2.

As shown above, the immunity detecting kit and the using method of immunity detecting kit of the present disclosure can be applied to detect the immunity of the subjects suffering from chronic inflammation so as to screen the Chinese herbal extracts having the immunity-suppressing effect from the Chinese herb combination in response to the overactive immune system, and thus it is favorable for formulating the treatment strategy.

### III. Analysis of the Effects of the Different Chinese Herbal Extracts and Nutritional Products on the Immunity of the Health Subjects

In the present experiment, the blood samples of 97 health subjects (those who do not have any disease records within three years) are analyzed by the using method 100 of immunity detecting kit and the using method 200 of immunity detecting kit of the present disclosure so as to compare the first results with the second results of the blood samples of the health subjects and then analyze the effects of the Chinese herbal extracts of the Chinese herb combination and the nutritional products of the nutrient combination on the immunity of the health subjects.

In the present experiment, the immune-stimulant agent of the immunity detecting kit is the phorbol 12-myristate 13-acetate, and the immunity detecting kit further includes the endotoxin inhibitor, wherein the concentration of the phorbol 12-myristate 13-acetate is 50 ng/ml, the endotoxin inhibitor is the polymyxin B with the concentration of 50 μg/mL, the concentration of each of nutritional products of the nutrient combination is 100 μg/mL, and the concentration of each of the Chinese herbal extracts of the Chinese herbal composition is 100 μg/mL. Further, the first culturing time is 24 hours or 48 hours, the second culturing time is 24 hours or 48 hours, the cytokine of the first culturing mixture includes TNF-α, IFN-γ and IL-2, and the cytokine of the second culturing mixture includes TNF-α, IFN-γ and IL-2. Moreover, the first culturing time and the second culturing time of each of the subjects are the same so as to obtain a reliable analytical result. The definitions of the first culturing time and the second culturing time are shown in the foregoing description and will not be described herein.

Reference is made to FIG. 10A and FIG. 10B. FIG. 10A shows the average expressed amounts of IFN-γ, TNF-α and IL-2 of the health subjects in response to different Chinese herbal extracts. FIG. 10B shows the average expressed amounts of IFN-γ, TNF-α and IL-2 of the health subjects in response to different Chinese herbal extracts and nutritional products. In detail, the numbers shown in the horizontal axis of each of FIG. 10A and FIG. 10B represent the Chinese herbal extracts and the nutritional products shown in Table 8 and Table 9, and the dashed line shown in each of FIG. 10A and FIG. 10B is the baseline according to the first result (Number 1). Further, “Basement” shown in Table 8 represents the group by treating the immune cells with the phorbol 12-myristate 13-acetate and the endotoxin inhibitor.

TABLE 8

| No. | Name | No. | Name |
|---|---|---|---|
| 1 | Basement | 2 | Codonopsis Radix |
| 3 | Atractylodis Macrocephalae | 4 | Poria |
| | Rhizoma | | |
| 5 | Glycyrrhizae Radix et | 6 | Angelicae Sinensis Radix |
| | Rhizoma | | |
| 7 | Chuanxiong Rhizoma | 8 | Paeoniae Radix Alba |
| 9 | Rehmanniae Radix Praeparata | 10 | Astragali Radix |
| 11 | Cinnamomi Cortex | 12 | Zingiberis Rhizoma |
| 13 | Curcumae Longae Rhizoma | 14 | Ginseng Radix et Rhizoma |
| 15 | Panacis Quinquefolii Radix | 16 | Salviae Miltiorrhizae |
| | | | Radix et Rhizoma |
| 17 | Jujubae Fructus | 18 | Lycii Fructus |
| 19 | Achyranthis Bidentatae | 20 | Polyporus |
| | Radix | | |
| 21 | Polygonati Rhizoma | 22 | Reynoutriae Multiflorae |
| | | | Radix |
| 23 | Dioscoreae Rhizoma | 24 | Saposhnikoviae Radix |
| 25 | Schisandrae Chinensis Fructus | 26 | Scutellariae Barbatae Herba |
| 27 | Spreading Hedyotis Herb | 28 | Si-Jun-Zi-Tang |
| 29 | Si-Wu-Tang | 30 | Shi-Quan-Da-Bu-Tang |
| 31 | Bu-Zhong-Yi-Qi-Tang | 32 | Sheng-Mai-San |
| 33 | Shen-Ling-Bai-Zhu-San | 34 | Sheng-Yu-Tang |
| 35 | Gui-Pi-Tang | 36 | Ren-Shen-Yang-Rong-Tang |
| 37 | Liu-Wei-Di-Huang-Wan | 38 | Yu-Ping-Feng-San |
| 39 | Jia-Wei-Xiao-Yao-San | 41 | Tremella fuciformis |
| 42 | Antrodia cinnamomea | 43 | Cordyceps |
| 44 | Ganoderma | 45 | Massa Medicata Fermentata |
| 46 | Scutellariae Radix | 47 | Ping-Wei-San |
| 48 | Siang-Sha-Liu-Jun-Zi-Tang | 49 | Trachelospermi Caulis cum |
| | | | Folium |
| 50 | Phellodendri Cortex | 51 | Zhi-Bo-Di-Huang-Wan |
| 52 | You-Gui-Wan | | |

TABLE 9

| No. | Name | No. | Name |
|---|---|---|---|
| 40 | Grifola frondose + | 53 | Chicken broth concentrate |
| | Curcumae Longae Rhizoma | | |
| 54 | Clam essence | 55 | Si-Wu drink |
| 56 | Vitamin B | 57 | Fucoidan |
| 58 | Cyanobacteria extract | 59 | Catechin |
| 60 | Soy isoflavone | 61 | Cranberry extract |
| 62 | Vitamin C | | |

As shown in FIG. 10A, FIG. 10B, Table 8 and Table 9, the Atractylodis Macrocephalae Rhizoma extract (Number 3), the Angelicae Sinensis Radix extract (Number 6), the Si-Jun-Zi-Tang (Number 28), the Si-Wu-Tang (Number 29), the Shi-Quan-Da-Bu-Tang (Number 30), the Zhi-Bo-Di-Huang-Wan (Number 51) and the cyanobacteria extract (Number 58) can significantly improve the average expressed amount of IFN-γ of the health subjects, the Codonopsis Radix extract (Number 2), the Atractylodis Macrocephalae Rhizoma extract (Number 3), the Poria extract (Number 4) and the Si-Wu-Tang (Number 29) can significantly improve the average expressed amount of TNF-α of the health subjects, and the cranberry extract (Number 61) can significantly improve the average expressed amount of IL-2 of the health subjects. Further, the Si-Wu drink (Number 55) listed in Table 9 is prepared by adding the additives such as rock sugar, Gellan gum and citric acid to the Si-Wu-Tang so as to analyze the immune activity of the conventional Si-Wu drink in the market for the health subjects.

Reference is made to FIG. 11A and FIG. 11B. FIG. 11A shows the sum of the average expressed amounts of IFN-γ, TNF-α and IL-2 of the health subjects in response to different Chinese herbal extracts. FIG. 11B shows the sum of the average expressed amounts of IFN-γ, TNF-α and IL-2 of the health subjects in response to different Chinese herbal extracts and nutritional products. FIG. 11A and FIG. 11B are obtained by respectively summarizing the results of the expressed amounts of cytokine of each of the Chinese herbal extracts in FIG. 10A and FIG. 10B. Further, the numbers and the corresponding Chinese herbal extracts shown in FIG. 11A and FIG. 11B are respectively the same as those shown in FIG. 10A and FIG. 10B and will not be described herein, and the dashed line shown in each of FIG. 11A and FIG. 11B is the baseline according to the first result (Number 1).

As shown in FIG. 11A, FIG. 11B, Table 8 and Table 9, the Codonopsis Radix extract (Number 2), the Atractylodis Macrocephalae Rhizoma extract (Number 3), the Poria extract (Number 4), the Angelicae Sinensis Radix extract (Number 6), the Zingiberis Rhizoma extract (Number 12), the Si-Wu-Tang (Number 29), the Ren-Shen-Yang-Rong-Tang (Number 36), the clam essence (Number 54) and the cranberry extract (Number 61) can significantly improve the sum of the average expressed amounts of TNF-α, IFN-γ and IL-2.

As shown above, the immunity detecting kit and the using method of immunity detecting kit of the present disclosure can be applied to detect the immunity of the health subjects so as to screen the Chinese herbal extracts or the nutritional products suitable for the current immune status of the subject from the Chinese herb combination or the nutrient combination so as to facilitate the formulation of the treatment strategy.

Therefore, by analyzing the change of the immune response of the immune cells in response to the Chinese herbal extracts of the Chinese herb combination or the nutritional products of the nutrient combination present disclosure, the immunity detecting kit and the using method of immunity detecting kit of the present disclosure can be used to screen the Chinese herbal extracts or the nutritional products having an immunity enhancing activity or an immunity suppressing activity based on the physical condition of the individual. Hence, the immunity detecting kit and the using method of immunity detecting kit of the present disclosure can provide the reference to selecting the Chinese herbal extracts and the nutritional products and have the application potential in the related arts.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fall within the scope of the following claims.

What is claimed is:

1. An immunity detecting kit, comprising:
an immune-stimulant agent comprising a phorbol 12-myristate 13-acetate (PMA) and a lipopolysaccharide (LPS); and
a Chinese herb combination comprising at least one Chinese herb extract, and the at least one Chinese herb extract comprising:
a Scutellariae Radix (Huangqin) extract, a Massa Medicata Fermentata (Shenqu) extract, a Spreading Hedyotis Herb (Baihuasheshecao) extract, a Scutellariae Barbatae Herba (Banzhilian) extract, a Schisandrae Chinensis Fructus (Wuweizi) extract, a Saposhnikoviae Radix (Fangfeng) extract, a Dioscoreae Rhizoma (Shanyao) extract, a Reynoutriae Multiflorae Radix (Heshouwu) extract, a Polygonati Rhizoma (Huangjing) extract, a Polyporus (Zhuling) extract, an Achyranthis Bidentatae Radix (Niuxi) extract, a Lycii Fructus (Gouqizi) extract, a Jujubae Fructus (Dazao) extract, a Salviae Miltiorrhizae Radix et Rhizoma (Danshen) extract, a Panacis Quinquefolii Radix (Xiyangshen) extract, a Ginseng Radix et Rhizoma (Renshen) extract, a Curcumae Longae Rhizoma (Jianghuang) extract, a Zingiberis Rhizoma (Ganjiang) extract, a Cinnamomi Cortex (cinnamon bark) extract, an Astragali Radix (Huangqi) extract, a Rehmanniae Radix Praeparata (Shudihuang) extract, a Paeoniae Radix Alba (Baishao) extract, a Chuanxiong Rhizoma (Chuanxiong) extract, an Angelicae Sinensis Radix (Danggui) extract, a Glycyrrhizae Radix et Rhizoma (Gancao) extract, a Poria (Fuling) extract, an Atractylodis Macrocephalae Rhizoma (Baizhu) extract, a Codonopsis Radix (Dangshen) extract, a Cyathulae Radix (Chuanniuxi) extract, an Anemarrhenae Rhizoma (Zhimu) extract, a Moutan Radicis Cortex (Mudanpi) extract, a Lycii Radicis Cortex (Digupi) extract, a Siegesbeckiae Herba (Xixiancao) extract, a Taxilli Herba (Sangjisheng) extract, a Gentianae Macrophyllae Radix (Qinjiao) extract, an Artemisiae Scopariae Herba (Yinchen) extract, a Reynoutriae Rhizoma et Radix (Huzhang) extract, a Plantaginis Semen (Checianzi) extract, a Taraxaci Herba (Pugongying) extract, a Lonicerae Japonicae Flos (Jinyinhua) extract, a Houttuyniae Herba (Yuxingcao) extract, an Isatidis Radix (Banlangen) extract, a Rhei Radix et Rhizoma (Dahuang) extract, a Cassiae Semen (Juemingzi) extract, a Phellodendri Cortex (Huangbo) extract, an Isatidis Folium (Daqingye) extract, a Gentianae Radix et Rhizoma (Longdan) extract, a Forsythiae Fructus (Lianqiao) extract, a Coptidis Rhizoma (Huanglian) extract, a Gardeniae Fructus (Zhizi) extract, a Tremella fuciformis extract, a Trachelospermi Caulis cum Folium (Luoshiteng) extract, a Ganoderma (Lingzhi) extract, an Antrodia cinnamomea extract and a Cordyceps (Dongchongxiacao) extract.

2. The immunity detecting kit of claim 1, wherein the Chinese herb combination further comprising:
a Long-Dan-Xie-Gan-Tang, a Yin-Chen-Hao-Tang, a Ban-Xia-Xie-Xin-Tang, a San-Huang-Xie-Xin-Tang, a Si-Jun-Zi-Tang, a Si-Wu-Tang, a Shi-Quan-Da-Bu-Tang, a Bu-Zhong-Yi-Qi-Tang, a Sheng-Mai-San, a Shen-Ling-Bai-Zhu-San, a Sheng-Yu-Tang, a Gui-Pi-Tang, a Ren-Shen-Yang-Rong-Tang, a Liu-Wei-Di-Huang-Wan, a Yu-Ping-Feng-San, a Jia-Wei-Xiao-Yao-San, a Ping-Wei-San, a Siang-Sha-Liu-Jun-Zi-Tang, a Zhi-Bo-Di-Huang-Wan and a You-Gui-Wan.

3. The immunity detecting kit of claim 1, further comprising:
an endotoxin inhibitor comprising a polymyxin B (PMB).

4. A using method of immunity detecting kit, comprising:
performing a sample processing step, wherein a blood sample is preprocessed so as to obtain a blood-cell isolate, and the blood-cell isolate comprises a plurality of immune cells;
providing the immunity detecting kit of claim 1;
performing a culturing step, wherein a part of the plurality of immune cells is treated with the immune-stimulant agent for a first culturing time so as to obtain a first culturing mixture, and another part of the plurality of immune cells is treated with the immune-stimulant agent and the Chinese herb combination for a second culturing time so as to obtain a second culturing mixture;
performing a detecting step, wherein an expressed amount of cytokine of the first culturing mixture is analyzed so as to obtain a first result, and an expressed amount of cytokine of the second culturing mixture is analyzed so as to obtain a second result; and
performing an analyzing step, wherein the first result is compared with the second result so as to assess an immune activity of the Chinese herb combination for the plurality of immune cells;
wherein when the second result is larger than or equal to the first result, the Chinese herb combination has an immunity enhancing activity for the plurality of immune cells;
wherein when the second result is smaller than the first result, the Chinese herb combination has an immunity suppressing activity for the plurality of immune cells.

5. The using method of immunity detecting kit of claim 4, wherein each of the plurality of immune cells is a monocyte.

6. The using method of immunity detecting kit of claim 4, wherein a concentration of the Chinese herb combination is 50 μg/mL to 200 μg/mL, and a concentration of the immune-stimulant agent is 50 ng/ml to 200 ng/mL.

7. The using method of immunity detecting kit of claim 4, wherein the first culturing time is 24 hours to 48 hours, and the second culturing time is 24 hours to 48 hours.

8. The using method of immunity detecting kit of claim 4, wherein when the immune-stimulant agent is the phorbol 12-myristate 13-acetate, the another part of the plurality of immune cells is treated with the immune-stimulant agent, the Chinese herb combination and an endotoxin inhibitor so as to obtain the second culturing mixture.

9. An immunity detecting kit, comprising:
an immune-stimulant agent comprising a phorbol 12-myristate 13-acetate and a lipopolysaccharide; and
a nutrient combination comprising at least one nutritional product, and the at least one nutritional product comprising:
a chicken broth concentrate, a clam essence, a cyanobacteria extract, a soy isoflavone, a cranberry extract, a catechin, a Grifola frondosa extract, a fucoidan, a vitamin B and a vitamin C.

10. The immunity detecting kit of claim 9, further comprising:
an endotoxin inhibitor comprising a polymyxin B.

11. A using method of immunity detecting kit, comprising:
performing a sample processing step, wherein a blood sample is preprocessed so as to obtain a blood-cell isolate, and the blood-cell isolate comprises a plurality of immune cells;
providing the immunity detecting kit of claim 9;
performing a culturing step, wherein a part of the plurality of immune cells is treated with the immune-stimulant agent for a first culturing time so as to obtain a first culturing mixture, and another part of the plurality of immune cells is treated with the immune-stimulant agent and the nutrient combination for a second culturing time so as to obtain a second culturing mixture;
performing a detecting step, wherein an expressed amount of cytokine of the first culturing mixture is analyzed so as to obtain a first result, and an expressed amount of cytokine of the second culturing mixture is analyzed so as to obtain a second result; and
performing an analyzing step, wherein the first result is compared with the second result so as to assess an immune activity of the nutrient combination for the plurality of immune cells;
wherein when the second result is larger than or equal to the first result, the nutrient combination has an immunity enhancing activity for the plurality of immune cells;
wherein when the second result is smaller than the first result, the nutrient combination has an immunity suppressing activity for the plurality of immune cells.

12. The using method of immunity detecting kit of claim 11, wherein each of the plurality of immune cells is a monocyte.

13. The using method of immunity detecting kit of claim 11, wherein a concentration of the nutrient combination is 50 μg/mL to 200 μg/mL, and a concentration of the immune-stimulant agent is 50 ng/ml to 200 ng/mL.

14. The using method of immunity detecting kit of claim 11, wherein the first culturing time is 24 hours to 48 hours, and the second culturing time is 24 hours to 48 hours.

15. The using method of immunity detecting kit of claim 11, wherein when the immune-stimulant agent is the phorbol 12-myristate 13-acetate, the another part of the plurality of immune cells is treated with the immune-stimulant agent, the nutrient combination and an endotoxin inhibitor so as to obtain the second culturing mixture.
